(19) United States
(12) Patent Application Publication
(10) Pub. No.: US 2022/0133727 A1
(43) Pub. Date: May 5, 2022

(54) COMBINATION THERAPIES FOR USE IN TREATING CANCER

(71) Applicant: Les Laboratoires Servier SAS, Suresnes Cedex (FR)

(72) Inventors: Marc Lee HYER, Cambridge, MA (US); Petar KALEV, Cambridge, MA (US); Katya MARJON, Cambridge, MA (US); Kevin MARKS, Cambridge, MA (US)

(21) Appl. No.: 17/430,346

(22) PCT Filed: Feb. 13, 2020
(86) PCT No.: PCT/US2020/018036

§ 371 Date: Aug. 12, 2021

Related U.S. Application Data
(60) Provisional application No. 62/805,179, filed on Feb. 13, 2019.

(51) Int. Cl. A61K 31/519; A61P 35/00; A61K 45/06; A61K 31/337; A61K 31/282; A61K 33/243; A61K 31/7068

(52) U.S. Cl. CPC A61K 31/519; A61P 35/00; A61K 45/06; A61K 31/7068; A61K 31/282; A61K 33/243; A61K 31/337

(57) ABSTRACT

The compound of Formula (I), or pharmaceutically acceptable salts thereof, is useful in, among other things, the treatment of MTAP-deficient lung cancer, such as NSCLC, or MTAP-deficient pancreatic cancer, such as PDAC, or MTAP-deficient esophageal cancer and provides a therapeutic advantage when used in combination with other agents as herein described compared to treatment with each agent when administered alone.

### CROSS-REFERENCE TO RELATED APPLICATIONS

This application claims the benefit of U.S. Provisional Patent Application No. 62/805,179, filed Feb. 13, 2019, the disclosure of which is incorporated by reference herein.

### FIELD OF THE INVENTION

The compound of Formula (I) and pharmaceutically acceptable salts thereof is useful in, among other things, the treatment of MTAP-deficient lung cancer, such as non-small cell lung cancer or NSCLC, or MTAP-deficient pancreatic cancer, such as pancreatic ductal adenocarcinoma or PDAC, or MTAP-deficient esophageal cancer and provides a therapeutic advantage when used in combination with other agents, as herein described, compared to treatment with each agent when administered alone.

### BACKGROUND

Methionine adenosyltransferase (MAT), which is also known as S-adenosylmethionine synthetase, is a cellular enzyme that catalyzes the synthesis of S-adenosyl methionine (SAM or AdoMet) from methionine and ATP; the catalysis is considered to be rate-limiting step of the methionine cycle. SAM is the propylamino donor in polyamine biosynthesis, the principal methyl donor for DNA methylation, and is involved in gene transcription and cellular proliferation as well as the production of secondary metabolites.

Two genes designated as MAT1A and MAT2A encode two distinct catalytic MAT isoforms, respectively. A third gene, MAT2B, encodes a MAT2A regulatory subunit. MAT1A is specifically expressed in the adult liver, whereas MAT2A is widely distributed. Because MAT isoforms differ in catalytic kinetics and regulatory properties, MAT1A-expressing cells have considerably higher SAM levels than do MAT2A-expressing cells. It has been found that hypomethylation of the MAT2A promoter and histone acetylation causes upregulation of MAT2A expression.

In hepatocellular carcinoma (HCC), the downregulation of MAT1A and the up-regulation of MAT2A occur, which is known as the MAT1A:MAT2A switch. The switch, accompanied with up-regulation of MAT2B, results in lower SAM contents, which provide a growth advantage to hepatoma cells. Because MAT2A plays a crucial role in facilitating the growth of hepatoma cells, it is a target for antineoplastic therapy. Recent studies have shown that silencing by using small interfering RNA substantially suppresses growth and induces apoptosis in hepatoma cells. See, e.g., T. Li et al., J. Cancer 7(10) (2016) 1317-1327.

Some cancer cell lines that are MTAP deficient are particularly sensitive to inhibition of MAT2A. Marjon et al. (Cell Reports 15(3) (2016) 574-587). MTAP (methylthioadenosine phosphorylase) is an enzyme widely expressed in normal tissues that catalyzes the conversion of methylthioadenosine (MTA) into adenine and 5-methylthioribose-1-phosphate. The adenine is salvaged to generate adenosine monophosphate, and the 5-methylthioribose-1-phosphate is converted to methionine and formate. Because of this salvage pathway, MTA can serve as an alternative purine source when de novo purine synthesis is blocked, e.g., with antimetabolites, such as L-alanosine.

MAT2A is dysregulated in additional cancers that lack MTAP-deletion, including hepatocellular carcinoma and leukemia. J. Cai et al., Cancer Res. 58 (1998) 1444-1450; T. S. Jani et al., Cell. Res. 19 (2009) 358-369. Silencing of MAT2A expression via RNA-interference results in anti-proliferative effects in several cancer models. H. Chen et al., Gastroenterology 133 (2007) 207-218; Q. Liu et al. Hepatol. Res. 37 (2007) 376-388.

Many human and murine malignant cells lack MTAP activity. MTAP deficiency is found not only in tissue culture cells but the deficiency is also present in primary leukemias, gliomas, melanomas, pancreatic cancers, non-small cell lung cancers (NSCLC), bladder cancers, astrocytomas, osteosarcomas, head and neck cancers, myxoid chondrosarcomas, ovarian cancers, endometrial cancers, breast cancers, soft tissue sarcomas, non-Hodgkin lymphoma, and mesotheliomas. The gene encoding for human MTAP maps to region 9p21 on human chromosome 9p. This region also contains the tumor suppressor genes p16INK4A (also known as CDKN2A) and pI5INK4B. These genes code for p16 and p15, which are inhibitors of the cyclin D-dependent kinases cdk4 and cdk6, respectively.

The p16INK4A transcript can alternatively be alternative reading frame (ARF) spliced into a transcript encoding p14ARF. p14ARF binds to MDM2 and prevents degradation of p53 (Pomerantz et al. (1998) Cell 92:713-723). The 9p21 chromosomal region is of interest because it is frequently homozygously deleted in a variety of cancers, including leukemias, NSLC, pancreatic cancers, gliomas, melanomas, and mesothelioma. The deletions often inactivate more than one gene. For example, Cairns et al. ((1995) Nat. Gen. 11:210-212) reported that after studying more than 500 primary tumors, almost all the deletions identified in such tumors involved a 170 kb region containing MTAP, p14ARF and P16INK4A. Carson et al. (WO 99/67634) reported that a correlation exists between the stage of tumor development and loss of homozygosity of the gene encoding MTAP and the gene encoding p16. For example, deletion of the MTAP gene, but not p16INK4A was reported to be indicative of a cancer at an early stage of development, whereas deletion of the genes encoding for p16 and MTAP was reported to be indicative of a cancer at a more advanced stage of tumor development. In some osteosarcoma patients, the MTAP gene was present at diagnosis but was deleted at a later time point (Garcia-Castellano et al., Clin. Cancer Res. 8(3) 2002 782-787).

International Application No. PCT/US2017/049439, which published as WO 2018/045071, describes novel MAT2A inhibitors, including 3-(cyclohex-1-en-1-yl)-6-(4-methoxyphenyl)-2-phenyl-5-(pyridine-3-ylamino)pyrazolo[1,5-a]pyrimidin-7(4H)-one, as demonstrated by biochemical and cellular assays.

### SUMMARY

The compound, 3-(cyclohex-1-en-1-yl)-6-(4-methoxyphenyl)-2-phenyl-5-(pyridine-3-ylamino)pyrzolo[1,5-a]pyrimidin-7(4H)-one may be referred to herein as a compound of Formula (I):

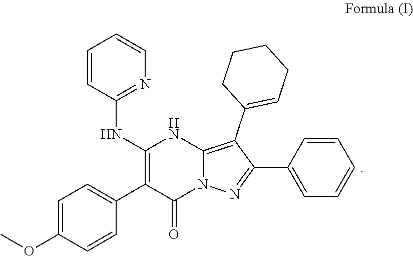 [SMILES: COc1ccc(-c2c(Nc3ccccn3)[nH]c3c(C4=CCCCC4)c(-c4ccccc4)nn3c2=O)cc1]

For ease of reference, the compound may also be referred to as Compound 1. The present disclosure also includes pharmaceutically acceptable salts of the compound of Formula (I).

The compound of Formula (I), or pharmaceutically acceptable salts thereof, is useful in, among other things, the treatment of lung cancer, such as NSCLC, or pancreatic cancer, such as PDAC, or esophageal cancer that are MTAP-deficient. In one embodiment, the compound of Formula (I) or a pharmaceutically acceptable salt thereof provides a therapeutic advantage when used in combination with at least one anti-mitotic agent in treating MTAP-deficient lung or MTAP-deficient pancreatic cancer, including MTAP-deficient NSCLC or MTAP-deficient PDAC or MTAP-deficient esophageal cancer. Relevant anti-mitotic agents include microtubule stabilizing agents and agents that disrupt the spindle assembly checkpoint. One example of an anti-mitotic agent is a taxane. Examples of taxanes include paclitaxel, nab-paclitaxel, or docetaxel, or alternative formulations thereof. In another embodiment, the anti-mitotic agent is an Aurora kinase inhibitor, including an inhibitor of Aurora kinase A or Aurora kinase B. In a further aspect of the application, the compound of Formula (I) or a pharmaceutically acceptable salt thereof provides a therapeutic advantage when used in combination with a DNA synthesis inhibitor in the treatment of MTAP-deficient lung cancer, such as NSCLC, or MTAP-deficient pancreatic cancer, such as PDAC. One example of a DNA synthesis inhibitor is gemcitabine. In another embodiment, the compound of Formula (I) or a pharmaceutically acceptable salt thereof and the DNA synthesis inhibitor is further combined with a taxane in the treatment of MTAP-deficient pancreatic cancer, including PDAC. Examples of a taxane are docetaxel and paclitaxel including nanoparticle-albumin bound paclitaxel. In still further embodiments, the compound of Formula (I) or a pharmaceutically acceptable salt thereof and a taxane are believed to provide a therapeutic advantage when used in combination for the treatment of MTAP-deficient esophageal cancer. In a still further aspect, the compound of Formula (I), or pharmaceutically acceptable salts thereof, provides a therapeutic advantage when used in combination with at least one antimetabolite agent in treating MTAP-deficient lung or MTAP-deficient pancreatic cancer, including MTAP-deficient NSCLC or MTAP-deficient PDAC or MTAP-deficient esophageal cancer. In another embodiment, the compound of Formula (I), or pharmaceutically acceptable salts thereof, may provide a therapeutic advantage when used in combination with at least one antimetabolite agent in treating MTAP-deficient mesothelioma. One example of an antimetabolite agent is pemetrexed disodium (“pemetrexed”). In yet further embodiments, any of the foregoing treatment methods may incorporate one or more additional therapeutic agents as detailed herein.

### BRIEF DESCRIPTION OF THE DRAWINGS

FIG. 1 is an illustration of a cell cycle analysis in synchronized HCT116 MTAP^{-/-}, which demonstrates that a compound of Formula (I), or a pharmaceutically acceptable salt thereof, inhibits cell cycle progression.

FIG. 2 illustrates a Western blot analysis for levels of Aurora B and phospho-Ser10-H3 during cell cycle progression.

FIGS. 3A and 3B illustrate the results of an immunofluorescence analysis demonstrating that the compound of Formula (I) leads to increased γH2AX in HCT116 MTAP^{-/-} cells.

FIGS. 4A and 4B illustrate a DAPI staining analysis that demonstrates an increased number of micronuclei formation.

FIG. 5A illustrates the results of an immunofluorescence analysis that demonstrates mitotic defects upon treatment with a compound of Formula (I). FIG. 5B illustrates the results of an γH2AX staining analysis to demonstrate that a compound of Formula (I) induces DNA damage in HCT116 MTAP^{-/-} cells.

FIG. 6 illustrates Loewe Synergy Scores, as herein defined, displayed as a scatter plot and ranked by median score across the cell line panel as described.

FIG. 7 illustrates the results of a combination index assessment with the compound of Formula (I) and two different taxane compounds: docetaxel and paclitaxel. Synergy plots demonstrate the interaction of docetaxel and paclitaxel in combination with the compound of Formula (I) in H2122 and KP4 cell lines.

FIG. 8 illustrates the results of Example 4, a combination of the compound of Formula (I) and docetaxel therapy in a pancreatic KP4 Xenograft Model.

FIG. 9 illustrates the results of Example 5, a combination of the compound of Formula (I) and paclitaxel therapy in a pancreatic cancer Xenograft Model (PA0372) in Female BALB/c Nude mice.

FIG. 10 illustrates the results of Example 6, a combination of the compound of Formula (I) and paclitaxel therapy in a pancreatic PAX041 PDX Model.

FIG. 11 illustrates the results of Example 7, a combination of the compound of Formula (I) and gemcitabine therapy in a pancreatic PAX041 PDX Model.

FIG. 12 illustrates the results of Example 8, a combination of the compound of Formula (I) and gemcitabine therapy in a pancreatic PDX model (PAX001).

FIG. 13 illustrates the results of Example 9, a combination of the compound of Formula (I) and gemcitabine therapy in a pancreatic KP4 model.

FIG. 14 illustrates the results of Example 10, a combination of the compound of Formula (I) and docetaxel therapy in a NSCLC PDX model (LU6412).

FIG. 15 illustrates the results of Example 11, a combination of the compound of Formula (I) and docetaxel therapy in a NSCLC PDX model (CTG-1194).

FIG. 16 illustrates the results of Example 12, a combination of the compound of Formula (I) and paclitaxel therapy in a pancreatic PDX model (PAX001).

FIG. 17 illustrates the results of Example 13, a combination of the compound of Formula (I) and docetaxel therapy in an esophageal PDX model (ES2263).

FIG. 18 illustrates the results of Example 14, a combination of the compound of Formula (I) and gemcitabine therapy in a pancreatic PDX model (PAX041).

FIG. 19 illustrates the results of Example 15, a combination of the compound of Formula (I) and gemcitabine therapy in a pancreatic PDX model (PAX001).

FIG. 20 illustrates the results of Example 16, a combination of the compound of Formula (I) and gemcitabine therapy in a pancreatic xenograft tumor (KP4).

FIG. 21 illustrates the results of Example 18, a combination of the compound of Formula (I) and gemcitabine therapy in a NSCLC PDX model (LU6431).

### DETAILED DESCRIPTION

As noted hereinabove and elsewhere in the application, the compound of Formula (I), or pharmaceutically acceptable salts thereof, is useful in, among other things, the treatment of MTAP-deficient lung cancer, such as NSCLC, or MTAP-deficient pancreatic cancer, such as PDAC or in the treatment of MTAP-deficient esophageal cancer.

In one embodiment, the compound of Formula (I) or a pharmaceutically acceptable salt thereof may provide a therapeutic advantage when used in combination with at least one anti-mitotic agent in treating MTAP-deficient lung or MTAP-deficient pancreatic cancers, more particularly MTAP-deficient NSCLC or MTAP-deficient PDAC, or in the treatment of MTAP-deficient esophageal cancer. Relevant anti-mitotic agents include microtubule stabilizing agents and agents that disrupt the spindle assembly checkpoint. In some embodiments, the anti-mitotic agent is a taxane. In some embodiments, examples of a taxane include docetaxel and paclitaxel including nanoparticle-albumin bound paclitaxel (nab-paclitaxel). In other embodiments, the anti-mitotic agent is an Aurora kinase inhibitor, including an inhibitor of Aurora kinase A or Aurora kinase B.

In a further aspect of the application, the compound of Formula (I) or a pharmaceutically acceptable salt thereof provides a therapeutic advantage when used in combination with a DNA synthesis inhibitor in the treatment of MTAP-deficient lung cancer, such as NSCLC, or MTAP-deficient pancreatic cancer, such as PDAC. In some embodiments, the DNA synthesis inhibitor is gemcitabine. In another embodiment, the compound of Formula (I) or a pharmaceutically acceptable salt thereof and a DNA synthesis inhibitor is further combined with a taxane in the treatment of MTAP-deficient pancreatic cancer, such as PDAC. In some embodiments, examples of a taxane include docetaxel and paclitaxel including nanoparticle-albumin bound paclitaxel.

In another embodiment, the compound of Formula (I) or a pharmaceutically acceptable salt thereof provides a therapeutic advantage when used in combination with an anti-mitotic in the treatment of MTAP-deficient esophageal cancer. In some embodiments, the anti-mitotic is a taxane. In some embodiments, the taxanes include docetaxel and paclitaxel including nanoparticle-albumin bound paclitaxel. In yet another embodiment, in the treatment of MTAP-deficient esophageal cancer, the compound of Formula (I) or a pharmaceutically acceptable salt thereof and the taxane are used in further combination with a platinum-based chemotherapeutic. In some embodiments, the platinum-based chemotherapeutic is cisplatin, carboplatin and/or oxaliplatin. In other embodiments, the compound of Formula (I) or a pharmaceutically acceptable salt thereof and the taxane are used in further combination with a platinum-based chemotherapeutic and an antimetabolite agent. In some embodiments the antimetabolite agent is 5-fluorouracil and/or capecitabine.

In other embodiments, the compound of Formula (I) or a pharmaceutically acceptable salt thereof provides a therapeutic advantage when used in combination with an antimetabolite agent in the treatment of MTAP-deficient lung cancer or MTAP-deficient pancreatic cancer or MTAP-deficient esophageal cancer. In some embodiments, the MTAP-deficient lung cancer is NSCLC. In still other embodiments the NSCLC is advanced non-squamous NSCLC. In some embodiments the antimetabolite agent is a pemetrexed. In other embodiments, the compound of Formula (I) or a pharmaceutically acceptable salt thereof and pemetrexed are used in further combination with a platinum-based chemotherapeutic. In some embodiments, the platinum-based chemotherapeutic is cisplatin, carboplatin and/or oxaliplatin. In still other embodiments the compound of Formula (I) or a pharmaceutically acceptable salt thereof and pemetrexed are used in further combination with a platinum-based chemotherapeutic and a PD-L1 checkpoint inhibitor such as pembrolizumab.

In other embodiments, the compound of Formula (I) or a pharmaceutically acceptable salt thereof provides a therapeutic advantage when used in combination with an antimetabolite agent in the treatment of MTAP-deficient mesothelioma. In some embodiments the antimetabolite is pemetrexed. In other embodiments, the compound of Formula (I) or a pharmaceutically acceptable salt thereof and pemetrexed are used in further combination with a platinum-based chemotherapeutic. In some embodiments, the platinum-based chemotherapeutic is cisplatin, carboplatin and/or oxaliplatin.

In still additional embodiments of any of the foregoing methods of treatment, the compound of Formula (I) or a pharmaceutically acceptable salt thereof and the one or more additional therapeutic agents may be administered concurrently. In yet additional embodiments of any of the foregoing methods of treatment, the compound of Formula (I) or a pharmaceutically acceptable salt thereof and the one or more additional therapeutic agents may be administered sequentially. In still other embodiments of any of the foregoing methods of treatment, the compound of Formula (I) or a pharmaceutically acceptable salt thereof is administered orally. In further embodiments of any of the foregoing methods of treatment, the compound of Formula (I) a pharmaceutically acceptable salt thereof is administered once or twice daily.

### Definitions

The phrase ‘MTAP-deficient’ lung or ‘MTAP-deficient’ pancreatic cancer or ‘MTAP-deficient’ esophageal cancer refers to lung or pancreatic or esophageal cancer, which lack activity of the metabolic enzyme Methylthioadenosine Phosphorylase (MTAP). Thus, an MTAP-deficient lung or MTAP-deficient pancreatic cancer or MTAP-deficient esophageal cancer occurs where there is a failure to express the MTAP gene, which may be assessed by the absence of MTAP gene, the lack of MTAP protein expression, or by accumulation of MTAP substrate MTA. In some embodiments the term ‘MTAP-deficient’ is referred to as ‘MTAP-deleted’ and/or ‘MTAP-null’ and thus the terms may be used interchangeably. For example in some embodiments, an ‘MTAP-deleted’ or ‘MTAP-null’ lung or ‘MTAP-deleted’ or ‘MTAP-null’ pancreatic cancer or ‘MTAP-deleted’ or ‘MTAP-null’ esophageal cancer refers to chromosomal loss of the MTAP gene, resulting in full or partial loss of MTAP DNA which prevents expression of functional, full length MTAP protein. In some embodiments, an MTAP-deficient lung or MTAP-deficient pancreatic cancer is a lung or pancreatic cancer, such as NSCLC or PDAC, in which the MTAP gene has been deleted, lost, or otherwise deactivated. Similarly, an MTAP-deficient esophageal cancer is an esophageal cancer in which the MTAP gene has been deleted, lost, or otherwise deactivated. In some embodiments, an MTAP-deficient lung, such as NSCLC, or MTAP-deficient pancreatic cancer, such as PDAC is a lung or pancreatic cancer in which the MTAP protein has a reduced function or is functionally impaired as compared to a wild type MTAP gene. Similarly, in some embodiments an MTAP-deficient esophageal cancer is an esophageal cancer in which the MTAP protein has a reduced function or is functionally impaired as compared to a wild type MTAP gene. Accordingly, in an embodiment of the present disclosure, there is provided a method for treating a MTAP-deficient lung cancer, such as NSCLC, or MTAP-deficient pancreatic cancer, such as PDAC, or an MTAP-deficient esophageal cancer in a subject, wherein the lung or pancreatic or esophageal cancer is characterized by at least one of (i) a reduction or absence of MTAP expression; (ii) absence of the MTAP gene; and (iii) reduced function of MTAP protein, as compared to lung or pancreatic cancers where the MTAP gene and/or protein is present and fully functioning, or as compared to lung or pancreatic cancers with the wild type MTAP gene.

As used herein, a “pharmaceutically acceptable salt” is a pharmaceutically acceptable, organic or inorganic acid or base salt of a compound of the invention. Representative pharmaceutically acceptable salts include, e.g., alkali metal salts, alkali earth salts, ammonium salts, water-soluble and water-insoluble salts, such as the acetate, amsonate (4,4-diaminostilbene-2, 2-disulfonate), benzenesulfonate, benzonate, bicarbonate, bisulfate, bitartrate, borate, bromide, butyrate, calcium, calcium edetate, camsylate, carbonate, chloride, citrate, clavulariate, dihydrochloride, edetate, edisylate, estolate, esylate, fumarate, gluceptate, gluconate, glutamate, glycollylarsanilate, hexafluorophosphate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, 3-hydroxy-2-naphthoate, oleate, oxalate, palmitate, pamoate (1,1-methene-bis-2-hydroxy-3-naphthoate, einbonate), pantothenate, phosphate/diphosphate, picrate, polygalacturonate, propionate, p-toluenesulfonate, salicylate, stearate, subacetate, succinate, sulfate, sulfosalicylate, suramate, tannate, tartrate, teoclate, tosylate, triethiodide, and valerate salts. A pharmaceutically acceptable salt can have more than one charged atom in its structure. In such instance, the pharmaceutically acceptable salt can have multiple counterions. Thus, a pharmaceutically acceptable salt can have one or more charged atoms and/or one or more counterions.

The terms “treat,” “treating,” and “treatment” refer to the amelioration or eradication of a disease or symptoms associated with a disease. In certain embodiments, such terms refer to minimizing the spread or worsening of the disease resulting from the administration of one or more prophylactic or therapeutic agents to a patient with such a disease.

The terms “prevent,” “preventing,” and “prevention” refer to the prevention of or the delay in the onset, recurrence, or spread of the disease in a patient resulting from the administration of a prophylactic or therapeutic agent.

The terms “effective amount” refer to an amount of a compound of Formula (I) or other active ingredient sufficient to provide a therapeutic or prophylactic benefit in the treatment or prevention of a disease or to delay or minimize symptoms associated with a disease. Further, a therapeutically effective amount with respect to a compound of Formula (I) means that amount of therapeutic agent alone, or in combination with other therapies, that provides a therapeutic benefit in the treatment or prevention of a disease. The terms may encompass an amount that improves overall therapy, reduces or avoids symptoms or causes of disease, or enhances the therapeutic efficacy of or synergies with another therapeutic agent.

A “patient” or “subject” includes an animal, such as a human, cow, horse, sheep, lamb, pig, chicken, turkey, quail, cat, dog, mouse, rat, rabbit, or guinea pig. In accordance with some embodiments, the animal is a mammal such as a non-primate and a primate (e.g., monkey and human). In one embodiment, a patient is a human, such as a human neonate, infant, child, adolescent, or adult. In one embodiment, the patient is a pediatric patient, including a patient from birth to eighteen years of age. In one embodiment, the patient is an adolescent patient, where an adolescent is a patient between the ages of 12 to 17 years of age. In one embodiment, the patient is an adult patient. In yet another embodiment, the terms indicating patient age are used in accordance with applicable regulatory guidance, such as, for example, the guidance set forth by the US FDA, where neonates are birth to one month of age, infants are one month up to two years of age; children are two years up to twelve years of age; and adolescents are twelve years up to sixteen years of age.

“Inhibitor” means a compound that prevents or reduces the amount of synthesis of SAM. In an embodiment, an inhibitor binds to MAT2A.

The “therapeutically effective amount” of a compound of Formula (I), or a pharmaceutically acceptable salt thereof, that is administered may be governed by considerations such as the minimum amount necessary to exert a cytotoxic effect, or to inhibit MAT2A activity, or both. Such amount may be below the amount that is toxic to normal cells, or the patient as a whole. Generally, the initial therapeutically effective amount of a compound of Formula (I), or a pharmaceutically acceptable salt thereof, to be administered is in the range of about 0.01 to about 200 mg/kg or about 0.1 to about 20 mg/kg of patient body weight per day, with the typical initial range being about 0.3 to about 15 mg/kg/day. Oral unit dosage forms, such as tablets and capsules, may contain from about 1 mg to about 1000 mg of a compound of Formula (I), or a pharmaceutically acceptable salt thereof. In another embodiment, such dosage forms may contain from about 20 mg to about 800 mg of a compound of Formula (I), or a pharmaceutically acceptable salt thereof. In yet another embodiment, such dosage forms may contain about 20 mg, 25 mg, 50 mg, 100 mg, 150 mg, 200 mg, 250 mg, 300 mg, 350 mg, 400 mg, 450 mg, 500 mg, 550 mg, 600 mg, 650 mg, 700 mg, 750 mg, or 800 mg of a compound of Formula (I), or a pharmaceutically acceptable salt thereof. In another aspect, the dosage is measured as an amount corresponding to an amount of free form equivalent of the Compound of Formula (I). “Free-form equivalent,” as used herein, refers to that quantity of the Compound of Formula (I), whether present in free form (or free base form), or as a salt, that corresponds to a given quantity of free form compound of Formula (I). In a further aspect, administering a therapeutically effective amount of the compound of Formula (I) or a pharmaceutically acceptable salt thereof includes circumstances wherein the combination, i.e. the compound of Formula (I) or a pharmaceutical salt thereof and one or more additional therapeutic agents, is administered within a specific period and for a duration of time. In some embodiments, the dosage form comprising the compound of Formula (I) or a pharmaceutical salt thereof is given once per day. In other embodiments, the dosage form is given twice a day. As used herein the term “daily dosing” means a particular dosing schedule for the compound of Formula (I) or a pharmaceutically acceptable salt thereof that takes place within a twenty-four period.

The term “pemetrexed” as used herein refers to (2S)-2-[[4-[2-(2-amino-4-oxido-7H-pyrrolo[2,3-d]pyrimidin-5-yl)ethyl]benzoyl]amino]pentanedioc acid, having the following structure. “Pemetrexed” also includes pharmaceutically acceptable salts thereof such as pemetrexed disodium which is available as Alimta®.

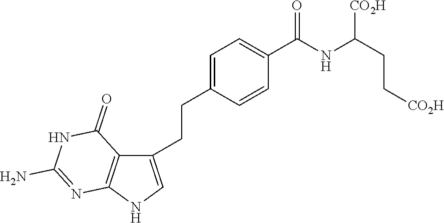 [SMILES: Nc1nc2[nH]cc(CCc3ccc(C(=O)NC(CCC(=O)O)C(=O)O)cc3)c2c(=O)[nH]1]

Positive therapeutic effects in cancer can be measured in a number of ways. The administration of a therapeutically effective amount of the combinations herein described are advantageous over the individual component compounds. As used herein “advantageous combinations” are those combinations that provide at least one of the following improved properties when compared to the individual administration of a therapeutically effective amount of a component compound: i) a greater anticancer effect than the most active single agent, alone; ii) synergistic anticancer effect; or iii) additive activity.

In some embodiments, synergy is determined using at least one of the models described herein. Combination effects may be characterized by comparing each data point to that of a combination reference model that was derived from the single agent curves. Three models are generally used: (1) the Highest Single Agent, which is a simple reference model where the expected combination effect is the maximum of the single agent responses at corresponding concentrations; (2) the Bliss Independence model, which represents the statistical expectation for independent competing inhibitors; (3) the Loewe Additivity model, which represents the expected response if both agents are actually the same compound; (4) the Chou-Talalay model, which estimates from dose-effect data of single and combined treatments and is represented as a Combination Index (CI) score; or a combination of one or more models.

The Loewe Additivity model is the most generally accepted reference for synergy, and, therefore, the Loewe Additivity model was used, and a metric was derived from it, which is characterized herein as the “Loewe Synergy Score.”

### Loewe Additivity Model

The Loewe additivity model is dose-based and applies only to the activity levels achieved by the single agents. Loewe Volume is used to assess the overall magnitude of the combination interaction in excess of the Loewe additivity model. Loewe Volume is particularly useful when distinguishing synergistic increases in a phenotypic activity (positive Loewe Volume) versus synergistic antagonisms (negative Loewe Volume). When antagonisms are observed, the Loewe Volume should be assessed to examine if there is any correlation between antagonism and a particular drug target-activity or cellular genotype. This model defines additivity as a non-synergistic combination interaction where the combination dose matrix surface should be indistinguishable from either drug crossed with itself. The calculation for Loewe additivity is:

I_{Loewe} that satisfies (X/X_{I})+(Y/Y_{I})=1

where X_{I} and Y_{I} are the single agent effective concentrations for the observed combination effect 1. For example, if 50% inhibition is achieved separately by 1 μM of drug A or 1 μM of drug B, a combination of 0.5 μM of A and 0.5 μM of B should also inhibit by 50%.

Activity observed in excess of Loewe additivity identifies a potential synergistic interaction. For the present analysis, empirically derived combination matrices were compared to their respective Loewe additivity models constructed from experimentally collected single agent dose response curves. Summation of this excess additivity across the dose response matrix is referred to as Loewe Volume. Positive Loewe volume suggests potential synergy, while negative Loewe Volume suggests potential antagonism.

### Loewe Synergy Score

To measure combination effects in excess of Loewe additivity, a scalar measure was devised to characterize the strength of synergistic interaction, which is herein termed the “Loewe Synergy Score.” The Loewe Synergy Score is calculated as:

Loewe Synergy Score=log ƒ_{X} log ƒ_{Y}Σmax(0,I_{data})(I_{data}−I_{Loewe})

The fractional inhibition for each component agent and combination point in the matrix is calculated relative to the median of all untreated/vehicle-treated control wells. The Loewe Synergy Score equation integrates the experimentally-observed activity volume at each point in the matrix in excess of a model surface numerically derived from the activity of the component agents using the Loewe model for additivity. Additional terms in the Loewe Synergy Score equation (above) are used to normalize for various dilution factors used for individual agents and to allow for comparison of synergy scores across an entire experiment. The inclusion of positive inhibition gating or an I_{data} multiplier removes noise near the zero effect level, and biases results for synergistic interactions at that occur at high activity levels. Combinations with higher maximum Growth Inhibition (GI) effects or those that are synergistic at low concentrations will have higher Loewe Synergy Scores.

As will be shown in the examples below, a further modified combination statistical analysis was performed to determine if the compound of Formula (I), when combined with an antimitotic agent or a DNA synthesis inhibitor, yielded anti-tumor combination benefit. The synergy score may be referred to as an “in vivo Synergy Score.”

In greater detail, in vivo methodology for this combination analysis is as follows: the input data consists of tumor volumes from each animal at successive time points. For each tumor volume, add 1 and take the log to base 10. For each animal, subtract the log(tumor volume+1) at the earliest time point from the log(tumor volume+1) at each time point. Use the resulting difference versus time data to calculate an area under the curve (AUC) value for each animal using the trapezoid rule. Calculate the mean AUC for each group. In vivo Synergy Score=100×(meanAUCAB−meanAUCA−meanAUCB+meanAUCV)/meanAUCV, where meanAUCAB, meanAUCA, meanAUCB and meanAUCV are the mean AUC values for the combination group, the A single agent group, the B single agent group and the vehicle/control group, respectively. Using the AUC values for the individual animals, carry out an ANOVA statistical test for whether the In vivo Synergy Score is not zero, obtaining a p value. For the combination to be considered synergistic the in vivo Synergy Score must be <0; an in vivo Synergy Score of 0 is exact additivity. As the in vivo Synergy Score increases above 0, the score moves away from additivity towards antagonism. If the p-value is above 0.05, the combination is considered to be additive. If the p-value is below 0.05 and the in vivo Synergy Score is less than zero, the combination is considered to be synergistic. If the p-value is below 0.05, the in vivo Synergy Score is greater than zero and the mean AUC for the combination is lower than the lowest mean AUC for the single agents, the combination is considered to be sub-additive. If the p-value is below 0.05, the in vivo Synergy Score is greater than zero and the mean AUC for the combination is greater than the mean AUC for at least one of the single agents, the combination is considered to be antagonistic.

### Chou-Talalay Model

An alternative model of synergy is the assessment of drug interactions using the Chou-Talalay model, which was introduced in 1983, and which allows an estimate the interactions between two drugs in combination studies, herein referred to as the “Combination Index (CI) Score.” According this model the interactions are estimated from dose-effect data of single and combined treatments and are represented as a Combination Index (CI) score. The CI is defined as (D1/ED×1)+(D2/ED×2), where ED×1 (or ED×2) is the dose of single agent drug 1 (or drug 2) which produces a selected effect x (such as 50% growth inhibition), and D1 and D2 are doses of drugs 1 and 2 which also produce the effect x when given in combination. For a given pair of compounds, multiple dose combinations were explored (in a matrix design) to identify the D1/D2 pair that give the lowest CI.

$$C I = \frac{{D}_{1}}{{( {D}_{m} )}_{1}} + \frac{{D}_{2}}{{( {D}_{m} )}_{2}} \qquad$$

If CI<1, the two drugs have a synergistic effect, and if CI>1, the drugs have an antagonistic effect. Lastly, a CI=1 suggests that the drugs have an additive effect. Reference is made to Chou, T. C. & Talalay, P. Quantitative analysis of dose-effect relationships: the combined effects of multiple drugs or enzyme inhibitors. Adv. Enzyme Regul. 22, 27-55 (1984).

### Pharmaceutical Compositions

The disclosure also provides a pharmaceutical composition comprising a therapeutically effective amount of a compound of Formula (I), or a pharmaceutically acceptable salt thereof, in admixture with a pharmaceutically acceptable carrier. In some embodiments, the composition further contains, in accordance with accepted practices of pharmaceutical compounding, one or more additional therapeutic agents, pharmaceutically acceptable excipients, diluents, adjuvants, stabilizers, emulsifiers, preservatives, colorants, buffers, flavor imparting agents.

The pharmaceutical composition of a compound of Formula (I), or a pharmaceutically acceptable salt thereof, is formulated, dosed, and administered in a fashion consistent with good medical practice. Factors for consideration in this context include the particular disorder being treated, the particular patient being treated, the clinical condition of the patient, the cause of the disorder, the site of delivery of the agent, the method of administration, the scheduling of administration, and other factors known to medical practitioners.

The pharmaceutical compositions may be administered orally, topically, parenterally, by inhalation or spray, or rectally in dosage unit formulations. The term parenteral as used herein includes subcutaneous injections, intravenous, intramuscular, or intrasternal injections, or infusion techniques.

Suitable oral compositions in accordance with the invention include without limitation tablets, troches, lozenges, aqueous or oily suspensions, dispersible powders or granules, emulsion, hard or soft capsules, syrups, or elixirs.

The pharmaceutical compositions may be suitable for single unit dosages that comprise a compound of Formula (I), or a pharmaceutically acceptable salt thereof, and a pharmaceutically acceptable carrier.

The pharmaceutical compositions suitable for oral use may be prepared according to any method known to the art for the manufacture of pharmaceutical compositions. For instance, liquid formulations may contain one or more agents selected from the group consisting of sweetening agents, flavoring agents, coloring agents, and preserving agents in order to provide pharmaceutically suitable and/or palatable preparations.

For tablet compositions, a compound of Formula (I), or a pharmaceutically acceptable salt thereof, may be formulated in admixture with non-toxic pharmaceutically acceptable excipients is used for the manufacture of tablets. Examples of such excipients include, without limitation, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known coating techniques to delay disintegration and absorption in the gastrointestinal tract and thereby to provide a sustained therapeutic action over a desired time period. For example, a time delay material such as glyceryl monostearate or glyceryl distearate may be employed.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin, or olive oil.

For aqueous suspensions, a compound of Formula (I), or a pharmaceutically acceptable salt thereof, may be admixed with excipients suitable for maintaining a stable suspension. Examples of such excipients include, without limitation, sodium carboxymethylcellulose, methylcellulose, hydropropylmethylcellulose, sodium alginate, polyvinylpyrrolidone, gum tragacanth, and gum acacia.

Oral suspensions can also contain dispersing or wetting agents, such as naturally-occurring phosphatide, for example, lecithin, or condensation products of an alkylene oxide with fatty acids, for example polyoxyethylene stearate, or condensation products of ethylene oxide with long chain aliphatic alcohols, for example, heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose or saccharin.

Oily suspensions may be formulated by suspending a compound of the present disclosure in a vegetable oil, for example arachis oil, olive oil, sesame oil, or coconut oil, or in a mineral oil such as liquid paraffin. The oily suspensions may contain a thickening agent, for example beeswax, hard paraffin, or cetyl alcohol.

Sweetening agents such as those set forth above, and flavoring agents may be added to provide palatable oral preparations. These compositions may be preserved by the addition of an anti-oxidant such as ascorbic acid.

Dispersible powders and granules suitable for preparation of an aqueous suspension by the addition of water provide a compound of the present disclosure in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

Pharmaceutical compositions of the present disclosure may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol, anhydrides, for example sorbitan monooleate, and condensation reaction products of the said partial esters with ethylene oxide, for example polyoxyethylene sorbitan monooleate. The emulsions may also contain sweetening and flavoring agents.

Syrups and elixirs may be formulated with sweetening agents, for example glycerol, propylene glycol, sorbitol, or sucrose. Such formulations may also contain a demulcent, a preservative, or flavoring and coloring agents. The pharmaceutical compositions may be in the form of a sterile injectable, an aqueous suspension, or an oleaginous suspension. This suspension may be formulated according to the known art using those suitable dispersing or wetting agents and suspending agents, which have been mentioned above. The sterile injectable preparation may also be sterile injectable solution or suspension in a non-toxic parentally acceptable diluent or solvent, for example as a solution in 1,3-butanediol. Among the acceptable vehicles and solvents that may be employed are water, Ringer's solution, and isotonic sodium chloride solution. In addition, sterile, fixed oils may be employed as a solvent or suspending medium. For this purpose, any bland fixed oil may be employed including synthetic mono- or diglycerides. In addition, fatty acids such as oleic acid may find use in the preparation of injectables.

The compound of Formula (I), or a pharmaceutically acceptable salt thereof, may also be administered in the form of suppositories for rectal administration of the drug. These compositions can be prepared by mixing the drug with a suitable non-irritating excipient that is solid at ordinary temperatures but liquid at the rectal temperature and will therefore melt in the rectum to release the drug. Such materials are cocoa butter and polyethylene glycols.

Compositions for parenteral administrations are administered in a sterile medium. Depending on the vehicle used and concentration the concentration of the drug in the formulation, the parenteral formulation can either be a suspension or a solution containing dissolved drug. Adjuvants such as local anesthetics, preservatives and buffering agents can also be added to parenteral compositions.

### Methods of Use

As noted hereinabove, the compound of Formula (I), or a pharmaceutically acceptable salt thereof, is useful in, for example, the treatment of lung cancer, such as NSCLC, or pancreatic cancer, such as PDAC, or esophageal cancer that are MTAP-deficient. In one embodiment, the compound of Formula (I), or a pharmaceutically acceptable salt thereof, provides a therapeutic advantage when used in combination with at least one anti-mitotic agent in treating MTAP-deficient lung cancer, such as NSCLC, or MTAP-deficient pancreatic cancer, such as PDAC or a MTAP-deficient esophageal cancer.

Relevant anti-mitotic agents include microtubule stabilizing agents and agents that disrupt the spindle assembly checkpoint. One example of an anti-mitotic agent is a taxane. One example of an anti-mitotic agent is an Aurora kinase inhibitor, including an inhibitor of Aurora kinase A or Aurora kinase B.

The disclosure also provides for the use of the compounds of Formula (I), or pharmaceutically acceptable salts thereof, in the treatment of mesothelioma. In some embodiments, the compounds of Formula (I), or pharmaceutically acceptable salts thereof, provide a therapeutic advantage when used in combination with an antimetabolite, when used in the treatment of MTAP-deficient mesothelioma. Relevant antimetabolites include pemetrexed or pharmaceutically acceptable salts thereof. In other embodiments, the compound of Formula (I) or a pharmaceutically acceptable salt thereof and pemetrexed are used in further combination with a platinum-based chemotherapeutic. In some embodiments, the platinum-based chemotherapeutic is carboplatin, oxaliplatin, nedaplatin, triplatin tetra nitrate, phenanthriplatin, picoplatin, or satraplatin. In other embodiments, the platinum-based chemotherapeutic is carboplatin or cisplatin.

In clinical practice, taxanes such as docetaxel and paclitaxel are frequently used in conjunction with other chemotherapeutic agents. For example, a nanoparticle-albumin bound paclitaxel (known as nab-paclitaxel or Abraxane®) is widely used in Pancreatic Ductal Adenocarcinoma (PDAC) in combination with the nucleoside analog DNA synthesis inhibitor gemcitabine (Gemzar®). Thus, in a further aspect, use of the compound of Formula (I) in combination with a DNA synthesis inhibitor and a taxane provides a therapeutic advantage in the treatment of MTAP-deficient pancreatic cancer such as PDAC. One example of a DNA synthesis inhibitor is gemcitabine. One example of a taxane is paclitaxel, including nanoparticle-albumin bound paclitaxel. Another example of a taxane is docetaxel.

In one embodiment, the method or use includes the treatment of MTAP-deficient lung cancer, such as non-small cell lung cancer (NSCLC), in a patient in need thereof comprising administering: (a) a therapeutically effective amount of a compound of Formula (I):

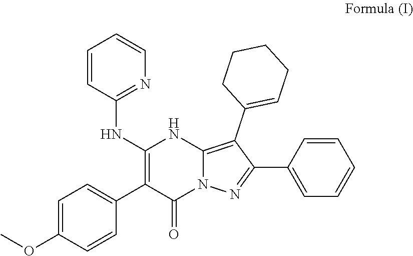 [SMILES: COc1ccc(-c2c(Nc3ccccn3)[nH]c3c(C4=CCCCC4)c(-c4ccccc4)nn3c2=O)cc1]

or a pharmaceutically acceptable salt thereof, and (b) a therapeutically effective amount of a taxane.

In one aspect, the taxane is docetaxel, paclitaxel, or nab-paclitaxel, or alternative formulations thereof. In one aspect, the taxane is docetaxel. In one aspect, the method or use further includes one or more additional therapeutic agents. In one aspect, the additional therapeutic agent is a platinum-based chemotherapeutic. In one aspect, the platinum-based chemotherapeutic is cisplatin, carboplatin, oxaplatin, nedaplatin, triplatin tetra nitrate, phenanthriplatin, picoplatin, or satraplatin. In one aspect, the platinum-based chemotherapeutic is carboplatin or cisplatin. In one aspect, the method of use further includes a therapeutically effective amount of a DNA synthesis inhibitor. In one aspect, the DNA synthesis inhibitor is gemcitabine. In one aspect, the lung cancer is MTAP-deleted or MTAP-null. In one aspect, the patient failed to respond, ceased responding, or experienced disease progression after one or more prior lines of therapy. In one aspect, the administration of the compound of Formula (I) or a pharmaceutically acceptable salt thereof is a second line of therapy for the treatment of MTAP-deficient lung cancer. In one aspect, the administration of the compound of Formula (I) or a pharmaceutically acceptable salt thereof is a third line of therapy for the treatment of MTAP-deficient lung cancer. In one aspect, the patient is newly diagnosed. In one aspect, the dosage of the compound of Formula (I) or a pharmaceutically acceptable salt thereof is about 20 mg to about 800 mg. In one aspect, the dosage is about 20 mg, 25 mg, 50 mg, 100 mg, 150 mg, 200 mg, 250 mg, 300 mg, 350 mg, 400 mg, 450 mg, 500 mg, 550 mg, 600 mg, 650 mg, 700 mg, 750 mg, or 800 mg. In one aspect, the dosage is selected from once or twice daily dosing. In one aspect, the administration is oral. In another aspect, the dosage is measured as an amount corresponding to an amount of free form equivalent of the Compound of Formula (I).

In one embodiment, the method or use includes the treatment of MTAP-deficient pancreatic cancer in a patient in need thereof comprising administering: (a) a therapeutically effective amount of a compound of Formula (I):

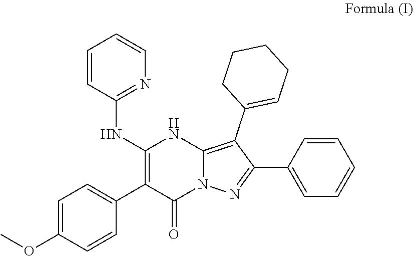 [SMILES: COc1ccc(-c2c(Nc3ccccn3)[nH]c3c(C4=CCCCC4)c(-c4ccccc4)nn3c2=O)cc1]

or a pharmaceutically acceptable salt thereof, and (b) a therapeutically effective amount of a taxane.

In one aspect, the taxane is paclitaxel, nab-paclitaxel, or docetaxel, or alternative formulations thereof. In one aspect, the taxane is nab-paclitaxel. In one aspect, the taxane is docetaxel. In one aspect, the method of use further includes a therapeutically effective amount of a DNA synthesis inhibitor. In one aspect, the DNA synthesis inhibitor is gemcitabine. In one aspect, the pancreatic cancer is MTAP-deleted or MTAP-null. In one aspect, the patient failed to respond, ceased responding, or experienced disease progression after one or more prior lines of therapy. In one aspect, the administration of the compound of Formula (I) or a pharmaceutically acceptable salt thereof is a second line of therapy for the treatment of MTAP-deficient pancreatic cancer. In one aspect, the administration of the compound of Formula (I) or a pharmaceutically acceptable salt thereof is a third line of therapy for the treatment of MTAP-deficient pancreatic cancer. In one aspect, the patient is newly diagnosed. In one aspect, the dosage of the compound of Formula (I) or a pharmaceutically acceptable salt thereof is about 20 mg to about 800 mg. In one aspect, the dosage is about 20 mg, 25 mg, 50 mg, 100 mg, 150 mg, 200 mg, 250 mg, 300 mg, 350 mg, 400 mg, 450 mg, 500 mg, 550 mg, 600 mg, 650 mg, 700 mg, 750 mg, or 800 mg. In another aspect, the dosage is measured as an amount corresponding to an amount of free form equivalent of the Compound of Formula (I). In one aspect, the dosage of the compound of Formula (I) or a pharmaceutically acceptable salt thereof is selected from once or twice daily dosing. In one aspect, the administration is oral. In one aspect, the MTAP-deficient pancreatic cancer is pancreatic ductal adenocarcinoma (PDAC). In one aspect, the MTAP-deficient pancreatic cancer is unresected, locally advanced or metastatic.

In one embodiment, the method or use includes treating a patient diagnosed with an MTAP-deficient lung or MTAP-deficient pancreatic cancer comprising administering: (a) a therapeutically effective amount of a compound of formula (I):

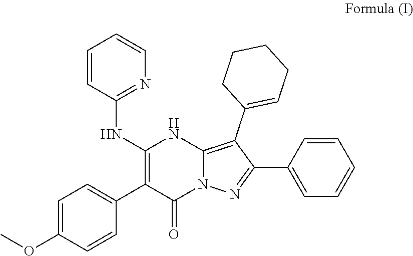 [SMILES: COc1ccc(-c2c(Nc3ccccn3)[nH]c3c(C4=CCCCC4)c(-c4ccccc4)nn3c2=O)cc1]

or a pharmaceutically acceptable salt thereof; and (b) at least one anti-mitotic agent.

In one aspect the anti-mitotic agent is a taxane. In one aspect the taxane is docetaxel, paclitaxel, or nab-paclitaxel, or alternative formulations thereof. In one aspect, the anti-mitotic agent is an Aurora kinase inhibitor. In one aspect, the Aurora kinase inhibitor is selective for Aurora kinase A or Aurora kinase B. In one aspect, the anti-mitotic targeted agent is ABT-348 or AZD1152. In one aspect, the MTAP-deficient pancreatic cancer is pancreatic ductal adenocarcinoma (PDAC). In one aspect, the MTAP-deficient pancreatic cancer is unresected, locally advanced, or metastatic. In one aspect, the MTAP-deficient lung cancer is non-small cell lung cancer. In one aspect, the MTAP-deficient lung cancer is squamous cell carcinoma or adenocarcinoma.

In one embodiment, the method or use includes treating a patient diagnosed with an MTAP-deficient lung or MTAP-deficient pancreatic cancer comprising administering: (a) a therapeutically effective amount of a compound of Formula (I):

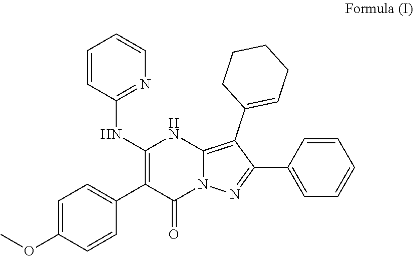 [SMILES: COc1ccc(-c2c(Nc3ccccn3)[nH]c3c(C4=CCCCC4)c(-c4ccccc4)nn3c2=O)cc1]

or a pharmaceutically acceptable salt thereof; and (b) at least one DNA synthesis inhibitor.

In one aspect, the DNA synthesis inhibitor is gemcitabine. In one aspect, the method or use further includes at least one taxane. In one aspect, the taxane is docetaxel, paclitaxel, or nab-paclitaxel, or alternative formulations thereof. In one aspect, the MTAP-deficient pancreatic cancer is pancreatic ductal adenocarcinoma (PDAC). In one aspect, the MTAP-deficient pancreatic cancer is unresected, locally advanced, or metastatic. In one aspect, the MTAP-deficient lung cancer is non-small cell lung cancer. In one aspect, the MTAP-deficient lung cancer is squamous cell carcinoma or adenocarcinoma.

In one embodiment, the method or use includes treating a patient diagnosed with an MTAP-deficient esophageal cancer comprising administering: (a) a therapeutically effective amount of a compound of Formula (I):

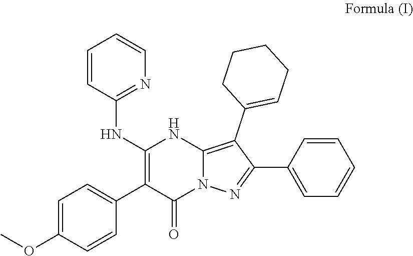 [SMILES: COc1ccc(-c2c(Nc3ccccn3)[nH]c3c(C4=CCCCC4)c(-c4ccccc4)nn3c2=O)cc1]

or a pharmaceutically acceptable salt thereof; and (b) at least one at least one anti-mitotic agent.

In one aspect the anti-mitotic agent is a taxane. In one aspect the taxane is docetaxel, paclitaxel, or nab-paclitaxel, or alternative formulations thereof. In another aspect the taxane is docetaxel or paclitaxel. In another aspect, the method or use further includes the administration of one or more additional therapeutic agents. In one aspect, the patient failed to respond, ceased responding, or experienced disease progression after one or more prior lines of therapy. In one aspect, the administration of the compound of Formula (I) or a pharmaceutically acceptable salt thereof is a second line of therapy for the treatment of MTAP-deficient esophageal cancer. In one aspect, the administration of the compound of Formula (I) or a pharmaceutically acceptable salt thereof is a third line of therapy for the treatment of MTAP-deficient esophageal cancer. In one aspect, the patient is newly diagnosed. In one aspect, the dosage of the compound of Formula (I) or a pharmaceutically acceptable salt thereof is about 20 mg to about 800 mg. In one aspect, the dosage is about 20 mg, 25 mg, 50 mg, 100 mg, 150 mg, 200 mg, 250 mg, 300 mg, 350 mg, 400 mg, 450 mg, 500 mg, 550 mg, 600 mg, 650 mg, 700 mg, 750 mg, or 800 mg. In one aspect, the dosage is selected from once or twice daily dosing. In one aspect, the administration is oral. In another aspect, the dosage is measured as an amount corresponding to an amount of free form equivalent of the Compound of Formula (I).

In further embodiments, the method or use includes treating a patient diagnosed with MTAP-deficient mesothelioma, comprising administering: (a) a therapeutically effective amount of a compound of Formula (I):

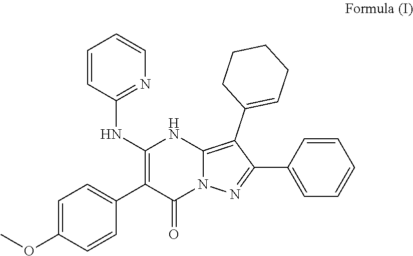 [SMILES: COc1ccc(-c2c(Nc3ccccn3)[nH]c3c(C4=CCCCC4)c(-c4ccccc4)nn3c2=O)cc1]

or a pharmaceutically acceptable salt thereof; and (b) pemetrexed disodium. In one aspect, the method or use further comprises administering one or more additional therapeutic agents. In another aspect, the additional therapeutic agent is a platinum-based chemotherapeutic. In a further aspect, the platinum-based chemotherapeutic is cisplatin, carboplatin, oxaliplatin, nedaplatin, triplatin tetra nitrate, phenanthriplatin, picoplatin, or satraplatin. In other aspects, the platinum-based chemotherapeutic is carboplatin or cisplatin. In one aspect, the patient failed to respond, ceased responding, or experienced disease progression after one or more prior lines of therapy. In one aspect, the administration of the compound of Formula (I) or a pharmaceutically acceptable salt thereof is a second line of therapy for the treatment of MTAP-deficient mesothelioma. In one aspect, the administration of the compound of Formula (I) or a pharmaceutically acceptable salt thereof is a third line of therapy for the treatment of MTAP-deficient mesothelioma. In one aspect, the patient is newly diagnosed. In one aspect, the dosage of the compound of Formula (I) or a pharmaceutically acceptable salt thereof is about 20 mg to about 800 mg. In one aspect, the dosage is about 20 mg, 25 mg, 50 mg, 100 mg, 150 mg, 200 mg, 250 mg, 300 mg, 350 mg, 400 mg, 450 mg, 500 mg, 550 mg, 600 mg, 650 mg, 700 mg, 750 mg, or 800 mg. In one aspect, the dosage is selected from once or twice daily dosing. In one aspect, the administration is oral. In another aspect, the dosage is measured as an amount corresponding to an amount of free form equivalent of the Compound of Formula (I).

For each of the embodiments and aspects, a further aspect includes wherein the compound of Formula (I) or a pharmaceutically acceptable salt thereof and the one or more additional therapeutic agents are administered concurrently. For each of the embodiments and aspects, a further aspect includes wherein the compound of Formula (I) or a pharmaceutically acceptable salt thereof and the one or more additional therapeutic agents are administered sequentially. For each of the embodiments and aspects, the method of use may further comprise radiation therapy.

One or more aspects and embodiments may be incorporated in a different embodiment although not specifically described. That is, all aspects and embodiments described herein may be combined in any way or combination.

### Aspects I

Aspect 1: A method for the treatment of MTAP-deficient non-small cell lung cancer (NSCLC) in a patient in need thereof comprising administering:

(a) a therapeutically effective amount of a compound of Formula (I):

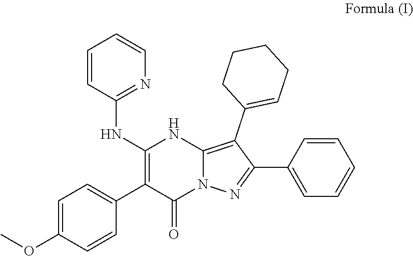 [SMILES: COc1ccc(-c2c(Nc3ccccn3)[nH]c3c(C4=CCCCC4)c(-c4ccccc4)nn3c2=O)cc1]

or a pharmaceutically acceptable salt thereof, and

(b) a therapeutically effective amount of a taxane.

Aspect 2: The method of Aspect 1, wherein the taxane is docetaxel, paclitaxel, or nab-paclitaxel, or alternative formulations thereof.

Aspect 3: The method of Aspect 2, wherein the taxane is docetaxel.

Aspect 4: The method of any one of Aspects 1-3, further comprising one or more additional therapeutic agents.

Aspect 5: The method of Aspect 4, wherein the additional therapeutic agent is a platinum-based chemotherapeutic.

Aspect 6: The method of Aspect 5, wherein the platinum-based chemotherapeutic is cisplatin, carboplatin, oxaplatin, nedaplatin, triplatin tetra nitrate, phenanthirplatin, piocplatin, or satraplatin.

Aspect 7: The method of Aspect 6, wherein the platinum-based chemotherapeutic is carboplatin or cisplatin.

Aspect 8: The method of any one of Aspects 1-7, wherein the NSCLC is MTAP-deleted or MTAP-null.

Aspect 9: The method of any one of Aspects 1-8, wherein the patient failed to respond, ceased responding, or experienced disease progression after one or more prior lines of therapy.

Aspect 10: The method of Aspect 9, wherein the administration is a second line of therapy.

Aspect 11: The method of Aspect 9, wherein the administration is a third line of therapy.

Aspect 12: The method of any one of Aspects 1-11, wherein the patient is newly diagnosed.

Aspect 13: The method of any one of Aspects 1-12, wherein the daily dosage of the compound of Formula (I) or pharmaceutically acceptable salt thereof is between about 20 mg to about 800 mg.

Aspect 14: The method of any one of Aspects 1-13, wherein the daily dosage is selected from once or twice daily dosing.

Aspect 15: The method of any one of Aspects 1-14, wherein the administration is oral.

Aspect 16: A method for the treatment of MTAP-deficient pancreatic cancer in a patient in need thereof comprising administering:

(a) a therapeutically effective amount of a compound of Formula (I):

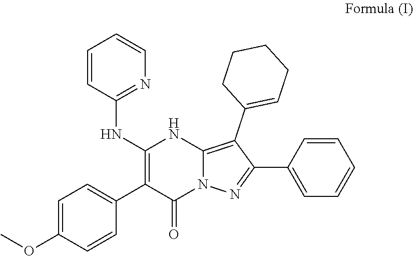 [SMILES: COc1ccc(-c2c(Nc3ccccn3)[nH]c3c(C4=CCCCC4)c(-c4ccccc4)nn3c2=O)cc1]

or a pharmaceutically acceptable salt thereof, and

(b) a therapeutically effective amount of a taxane.

Aspect 17: The method of Aspect 16, wherein the taxane is paclitaxel, nab-paclitaxel, or docetaxel, or alternative formulations thereof.

Aspect 18: The method of Aspect 17, wherein the taxane is nab-paclitaxel.

Aspect 19: The method of any one of Aspects 16-18, further comprising a therapeutically effective amount of a DNA synthesis inhibitor.

Aspect 20: The method of Aspect 19, wherein the DNA synthesis inhibitor is gemcitabine.

Aspect 21: The method of any one of Aspects 16-20, wherein the pancreatic cancer is MTAP-deleted or MTAP-null.

Aspect 22: The method of any one of Aspects 16-21, wherein the patient failed to respond, ceased responding, or experienced disease progression after one or more prior lines of therapy.

Aspect 23: The method of Aspect 22, wherein the administration is a second line of therapy.

Aspect 24: The method of Aspect 23, wherein the administration is a third line of therapy.

Aspect 25: The method of any one of Aspects 16-24, wherein the patient is newly diagnosed.

Aspect 26: The method of any one of Aspects 16-25, wherein the daily dosage of the compound of Formula (I) or pharmaceutically acceptable salt thereof is between about 20 mg to about 800 mg.

Aspect 27: The method of any one of Aspects 16-26, wherein the daily dosage is selected from once or twice daily dosing.

Aspect 28: The method of any one of Aspects 16-27, wherein the administration is oral.

Aspect 29: The method of any one of Aspects 16-28, wherein the pancreatic cancer is pancreatic ductal adenocarcinoma (PDAC).

Aspect 30: The method of any one of Aspects 16-29, wherein the pancreatic cancer is unresected, locally advanced or metastatic.

Aspect 31: A method of treating a patient diagnosed with an MTAP-deficient lung or MTAP-deficient pancreatic cancer comprising administering:

(a) a therapeutically effective amount of a compound of formula (I):

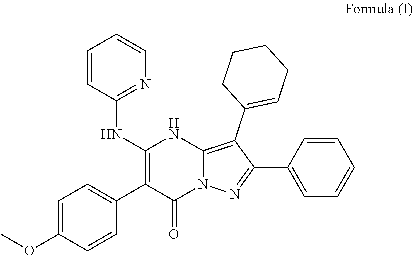 [SMILES: COc1ccc(-c2c(Nc3ccccn3)[nH]c3c(C4=CCCCC4)c(-c4ccccc4)nn3c2=O)cc1]

or a pharmaceutically acceptable salt thereof; and

(b) at least one anti-mitotic agent.

Aspect 32: The method of Aspect 31, wherein the anti-mitotic agent is an Aurora kinase inhibitor, or both.

Aspect 33: The method of Aspect 32, wherein the Aurora kinase inhibitor is selective for Aurora kinase A or Aurora kinase B.

Aspect 34: The method of Aspect 32 or 33, wherein the anti-mitotic targeted agent is ABT-348 or AZD1152.

Aspect 35: The method of any one of Aspects 31-34, wherein the pancreatic cancer is pancreatic ductal adenocarcinoma (PDAC).

Aspect 36: The method of any one of Aspects 31-35, wherein the pancreatic cancer is unresected, locally advanced, or metastatic.

Aspect 37: The method of Aspect 31, wherein the lung cancer is non-small cell lung cancer.

Aspect 38: The method of Aspect 37, wherein the lung cancer is squamous cell carcinoma or adenocarcinoma

Aspect 39: A method of treating a patient diagnosed with an MTAP-deficient lung or MTAP-deficient pancreatic cancer comprising administering:

(a) a therapeutically effective amount of a compound of formula (I):

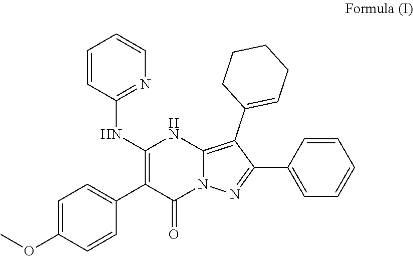 [SMILES: COc1ccc(-c2c(Nc3ccccn3)[nH]c3c(C4=CCCCC4)c(-c4ccccc4)nn3c2=O)cc1]

or a pharmaceutically acceptable salt thereof; and

(b) at least one DNA synthesis inhibitor.

Aspect 40: The method of Aspect 39, wherein the DNA synthesis inhibitor is gemcitabine.

Aspect 41: The method of Aspect 39 or 40, further comprising at least one taxane.

Aspect 42: The method of Aspect 41, wherein the taxane is docetaxel, paclitaxel, or nab-paclitaxel, or alternative formulations thereof.

Aspect 43: The method of any one of Aspects 39-42, wherein the pancreatic cancer is pancreatic ductal adenocarcinoma (PDAC).

Aspect 44: The method of any one of Aspects 39-43, wherein the pancreatic cancer is unresected, locally advanced, or metastatic.

Aspect 45: The method of Aspect 39, wherein the lung cancer is non-small cell lung cancer.

Aspect 46: The method of Aspect 45, wherein the lung cancer is squamous cell carcinoma or adenocarcinoma.

Aspect 47: The method of any one of Aspects 1-46, wherein the compound of Formula (I) and the one or more additional therapeutic agents are administered concurrently.

Aspect 48: The method of any one of Aspects 1-46, wherein the compound of Formula (I) and the one or more additional therapeutic agent are administered sequentially.

Aspect 49: The method of any one of Aspects 1-48, further comprising radiation therapy.

Aspect 50: A method for the treatment of MTAP-deficient esophageal cancer in a patient in need thereof comprising administering:

(a) a therapeutically effective amount of a compound of Formula (I):

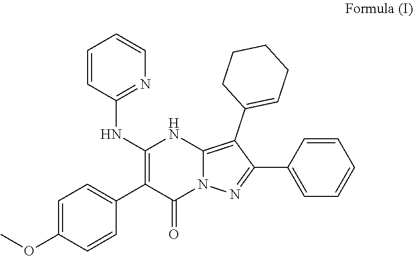 [SMILES: COc1ccc(-c2c(Nc3ccccn3)[nH]c3c(C4=CCCCC4)c(-c4ccccc4)nn3c2=O)cc1]

or a pharmaceutically acceptable salt thereof, and

(b) a therapeutically effective amount of a taxane.

Aspect 51: The method of Aspect 50, wherein the taxane is paclitaxel, nab-paclitaxel, or docetaxel, or alternative formulations thereof.

Aspect 52: The method of Aspect 51, wherein the taxane is docetaxel.

Aspect 53: The method of Aspect 50 wherein the esophageal cancer is MTAP-deleted or MTAP-null.

Aspect 54: The method of Aspect 50, wherein the patient failed to respond, ceased responding, or experienced disease progression after one or more prior lines of therapy.

Aspect 55: The method of Aspect 54, wherein the administration is a second line of therapy.

Aspect 56: The method of Aspect 54, wherein the administration is a third line of therapy.

Aspect 57: The method of Aspect 50, wherein the patient is newly diagnosed.

Aspect 58: The method of Aspect 50, wherein the daily dosage is selected from once or twice daily dosing.

Aspect 59: The method of Aspect 50, wherein the administration is oral.

### Aspects II

Aspect 1. A method for the treatment of MTAP-deficient non-small cell lung cancer (NSCLC) in a patient in need thereof, comprising administering:

(a) a therapeutically effective amount of a compound of Formula (I):

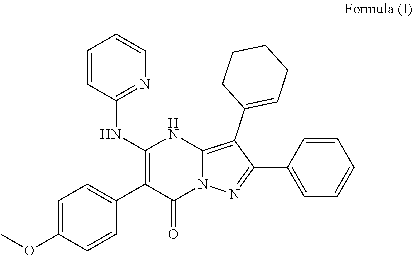 [SMILES: COc1ccc(-c2c(Nc3ccccn3)[nH]c3c(C4=CCCCC4)c(-c4ccccc4)nn3c2=O)cc1]

or a pharmaceutically acceptable salt thereof, and

(b) a therapeutically effective amount of a taxane.

Aspect 2. A compound of Formula (I), or a pharmaceutically acceptable salt thereof, for use in treating MTAP-deficient non-small cell lung cancer (NSCLC) in combination with a therapeutically effective amount of a taxane:

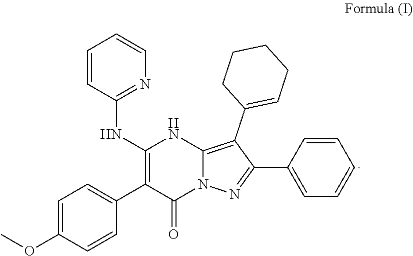 [SMILES: COc1ccc(-c2c(Nc3ccccn3)[nH]c3c(C4=CCCCC4)c(-c4ccccc4)nn3c2=O)cc1]

Aspect 3. The method of Aspect 1 or compound of Aspect 2, wherein the taxane is docetaxel, paclitaxel, or nab-paclitaxel.

Aspect 4. The method or compound of Aspect 2, wherein the taxane is docetaxel.

Aspect 5. The method of Aspect 1, 3, or 4, further comprising administering one or more additional therapeutic agents.

Aspect 6. The compound of any one of Aspects 2-4, wherein the combination further comprises one or more additional therapeutic agents.

Aspect 7. The method of Aspect 5 or compound of Aspect 6, wherein the additional therapeutic agent is a platinum-based chemotherapeutic.

Aspect 8. The method or compound of Aspect 7, wherein the platinum-based chemotherapeutic is cisplatin, carboplatin, oxaliplatin, nedaplatin, triplatin tetra nitrate, phenanthriplatin, picoplatin, or satraplatin.

Aspect 9. The method or compound of Aspect 7 or 8, wherein the platinum-based chemotherapeutic is carboplatin or cisplatin.

Aspect 10. The method of any one of Aspects 1, 3-5, or 7-9, wherein the patient failed to respond, ceased responding, or experienced disease progression after one or more prior lines of therapy for treating MTAP-deficient NSCLC.

Aspect 11. The method or compound of Aspect 10, wherein the compound of Formula (I) or a pharmaceutically acceptable salt thereof is a second line of therapy for treating MTAP-deficient NSCLC.

Aspect 12. The method or compound of Aspect 10, wherein the compound of Formula (I) or a pharmaceutically acceptable salt thereof is a third line of therapy for treating MTAP-deficient NSCLC.

Aspect 13. The method or compound of any one of Aspects 1-12, wherein the MTAP-deficient NSCLC is newly diagnosed.

Aspect 14. The method or compound of any one of Aspects 1-13, wherein the dosage of the compound of Formula (I) or a pharmaceutically acceptable salt thereof is about 20 mg to about 800 mg.

Aspect 15. The method or compound of any one of Aspects 1-14, wherein the dosage of the compound of Formula (I) or a pharmaceutically acceptable salt thereof is once or twice daily dosing.

Aspect 16. The method or compound of any one of Aspects 1-15, wherein the compound of Formula (I) or a pharmaceutically acceptable salt thereof is administered orally or formulated for oral administration.

Aspect 17. A method for the treatment of MTAP-deficient pancreatic cancer in a patient in need thereof, comprising administering:

(a) a therapeutically effective amount of a compound of Formula (I):

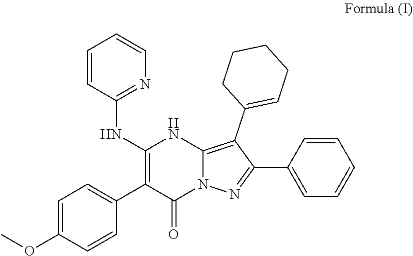 [SMILES: COc1ccc(-c2c(Nc3ccccn3)[nH]c3c(C4=CCCCC4)c(-c4ccccc4)nn3c2=O)cc1]

or a pharmaceutically acceptable salt thereof, and

(b) a therapeutically effective amount of a taxane.

Aspect 18. A compound of Formula (I), or a pharmaceutically acceptable salt thereof, for use in treating MTAP-deficient pancreatic cancer, in combination with a therapeutically effective amount of a taxane:

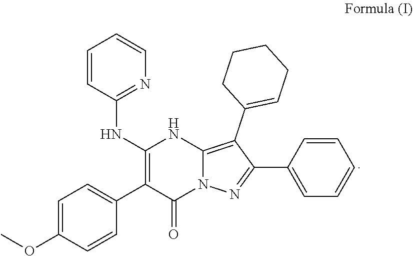 [SMILES: COc1ccc(-c2c(Nc3ccccn3)[nH]c3c(C4=CCCCC4)c(-c4ccccc4)nn3c2=O)cc1]

Aspect 19. The method of Aspect 17 or compound of Aspect 18, wherein the taxane is paclitaxel, nab-paclitaxel, or docetaxel.

Aspect 20. The method or compound of Aspect 19, wherein the taxane is nab-paclitaxel.

Aspect 21. The method of any one of Aspects 17, 19, or 20, further comprising administering a therapeutically effective amount of a DNA synthesis inhibitor.

Aspect 22. The compound of any one of Aspects 18-20, wherein the combination further comprises a therapeutically effective amount of a DNA synthesis inhibitor.

Aspect 23. The method or compound of Aspect 21 or 22, wherein the DNA synthesis inhibitor is gemcitabine.

Aspect 24. The method of any one of Aspects 17, 19-21, or 23, wherein the patient failed to respond, ceased responding, or experienced disease progression after one or more prior lines of therapy for treating MTAP-deficient pancreatic cancer.

Aspect 25. The method or compound of Aspect 24, wherein the compound of Formula (I) or a pharmaceutically acceptable salt thereof is a second line of therapy for treating MTAP-deficient pancreatic cancer.

Aspect 26. The method or compound of Aspect 25, wherein the compound of Formula (I) or a pharmaceutically acceptable salt thereof is a third line of therapy for treating MTAP-deficient pancreatic cancer.

Aspect 27. The method or compound of any one of Aspects 17-26, wherein the MTAP-deficient pancreatic cancer is newly diagnosed.

Aspect 28. The method or compound of any one of Aspects 17-27, wherein the dosage of the compound of Formula (I) or a pharmaceutically acceptable salt thereof is about 20 mg to about 800 mg.

Aspect 29. The method or compound of any one of Aspects 17-28, wherein the dosage of the compound of Formula (I) or a pharmaceutically acceptable salt thereof is selected from once or twice daily dosing.

Aspect 30. The method or compound of any one of Aspects 17-29, wherein the administration of the compound of Formula (I) or a pharmaceutically acceptable salt thereof is oral or the compound is formulated for oral administration.

Aspect 31. The method or compound of any one of Aspects 17-27, wherein the pancreatic cancer is pancreatic ductal adenocarcinoma (PDAC).

Aspect 32. The method or compound of any one of Aspects 17-31, wherein the pancreatic cancer is unresected, locally advanced or metastatic.

Aspect 33. The method of any one of Aspects 1, 3-5, 7-17, 19-21, or 23-32, wherein the compound of Formula (I) or a pharmaceutically acceptable salt thereof and the taxane are administered concurrently.

Aspect 34. The method of any one of Aspects 1, 3-5, 7-17, 19-21, or 23-32, wherein the compound of Formula (I) or a pharmaceutically acceptable salt thereof and the taxane are administered sequentially.

Aspect 35. A method of treating a patient diagnosed with a cancer that is an MTAP-deficient lung cancer, MTAP-deficient pancreatic cancer, or a MTAP-deficient esophageal cancer, comprising administering:

(a) a therapeutically effective amount of a compound of Formula (I):

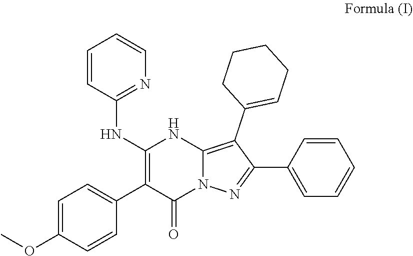 [SMILES: COc1ccc(-c2c(Nc3ccccn3)[nH]c3c(C4=CCCCC4)c(-c4ccccc4)nn3c2=O)cc1]

or a pharmaceutically acceptable salt thereof; and

(b) at least one anti-mitotic agent.

Aspect 36. A compound of Formula (I), or a pharmaceutically acceptable salt thereof, for use in treating a cancer that is an MTAP-deficient lung cancer, MTAP-deficient pancreatic cancer, or a MTAP-deficient esophageal cancer, in combination with at least one anti-mitotic agent:

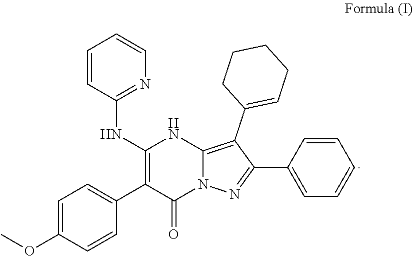 [SMILES: COc1ccc(-c2c(Nc3ccccn3)[nH]c3c(C4=CCCCC4)c(-c4ccccc4)nn3c2=O)cc1]

Aspect 37. The method of Aspect 35 or compound of Aspect 36, wherein the anti-mitotic agent is an Aurora kinase inhibitor.

Aspect 38. The method or compound of Aspect 37, wherein the Aurora kinase inhibitor is selective for Aurora kinase A or Aurora kinase B.

Aspect 39. The method or compound of Aspect 37 or 38, wherein the anti-mitotic targeted agent is ABT-348 or AZD1152.

Aspect 40. The method or compound of any one of Aspects 35-39, wherein the cancer is an MTAP-deficient pancreatic cancer.

Aspect 41. The method or compound of Aspect 40, wherein the MTAP-deficient pancreatic cancer is pancreatic ductal adenocarcinoma (PDAC).

Aspect 42. The method or compound of Aspect 40 or 41, wherein the pancreatic cancer is unresected, locally advanced, or metastatic.

Aspect 43. The method or compound of any one of Aspects 35-39, wherein the cancer is an MTAP-deficient lung cancer.

Aspect 44. The method or compound of Aspect 43, wherein the MTAP-deficient lung cancer is non-small cell lung cancer.

Aspect 45. The method or compound of Aspect 43 or 44, wherein the MTAP-deficient lung cancer is squamous cell carcinoma or adenocarcinoma.

Aspect 46. The method or compound of any one of Aspects 35-39, wherein the cancer is an MTAP-deficient esophageal cancer.

Aspect 47. A method of treating a patient diagnosed with a cancer that is an MTAP-deficient lung cancer, MTAP-deficient pancreatic cancer or MTAP-deficient esophageal cancer comprising administering:

(a) a therapeutically effective amount of a compound of Formula (I):

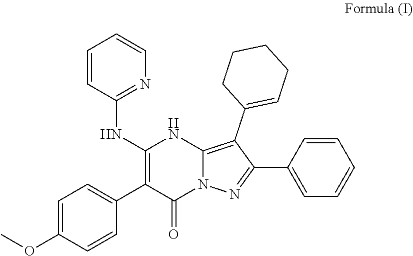 [SMILES: COc1ccc(-c2c(Nc3ccccn3)[nH]c3c(C4=CCCCC4)c(-c4ccccc4)nn3c2=O)cc1]

or a pharmaceutically acceptable salt thereof; and

(b) at least one DNA synthesis inhibitor.

Aspect 48. A compound of Formula (I), or a pharmaceutically acceptable salt thereof, for use in treating a cancer that is an MTAP-deficient lung cancer, MTAP-deficient pancreatic cancer or MTAP-deficient esophageal cancer, in combination with at least one DNA synthesis inhibitor:

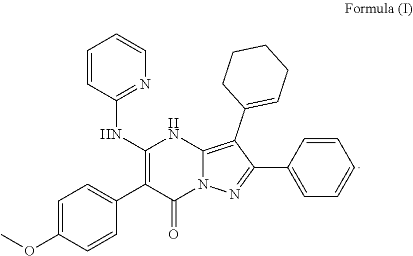 [SMILES: COc1ccc(-c2c(Nc3ccccn3)[nH]c3c(C4=CCCCC4)c(-c4ccccc4)nn3c2=O)cc1]

Aspect 49. The method of Aspect 47 or compound of Aspect 48, wherein the DNA synthesis inhibitor is gemcitabine.

Aspect 50. The method of Aspect 47 or 49, further comprising administering at least one taxane.

Aspect 51. The compound of Aspect 48, wherein the combination further comprises at least one taxane.

Aspect 52. The method or compound of Aspect 50 or 51, wherein the taxane is docetaxel, paclitaxel, or nab-paclitaxel.

Aspect 53. The method or compound of any one of Aspects 47-52, wherein the cancer is MTAP-deficient pancreatic cancer.

Aspect 54. The method or compound of Aspect 53, wherein the MTAP-deficient pancreatic cancer is pancreatic ductal adenocarcinoma (PDAC).

Aspect 55. The method or compound of Aspect 53 or 54, wherein the pancreatic cancer is unresected, locally advanced, or metastatic.

Aspect 56. The method or compound of any one of Aspects 47-52, wherein the cancer is MTAP-deficient lung cancer.

Aspect 57. The method or compound of Aspect 56, wherein the MTAP-deficient lung cancer is non-small cell lung cancer.

Aspect 58. The method or compound of Aspect 56 or 57, wherein the MTAP-deficient lung cancer is squamous cell carcinoma or adenocarcinoma.

Aspect 59. The method or compound of any one of Aspects 47-52 where in the cancer is MTAP-deficient esophageal cancer.

Aspect 60. The method of any one of Aspects 1, 3-5, 7-17, 19-21, 23-32, 35, 37-50, and 52, wherein the compound of Formula (I) or a pharmaceutically acceptable salt thereof and the DNA synthase inhibitor are administered concurrently.

Aspect 61. The method of any one of Aspects 1, 3-5, 7-17, 19-21, 23-32, 35, 37-50, and 52-59, wherein the compound of Formula (I) or a pharmaceutically acceptable salt thereof and the DNA synthase inhibitor are administered sequentially.

Aspect 62. The method or compound of any one of Aspects 1-61, further comprising radiation therapy.

Aspect 63. A method for the treatment of MTAP-deficient esophageal cancer in a patient in need thereof, comprising administering:

(a) a therapeutically effective amount of a compound of Formula (I):

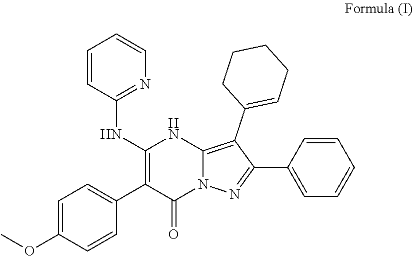 [SMILES: COc1ccc(-c2c(Nc3ccccn3)[nH]c3c(C4=CCCCC4)c(-c4ccccc4)nn3c2=O)cc1]

or a pharmaceutically acceptable salt thereof, and

(b) a therapeutically effective amount of a taxane.

Aspect 64. A compound of Formula (I), or a pharmaceutically acceptable salt thereof, for use in treating MTAP-deficient esophageal cancer, in combination with a therapeutically effective amount of a taxane:

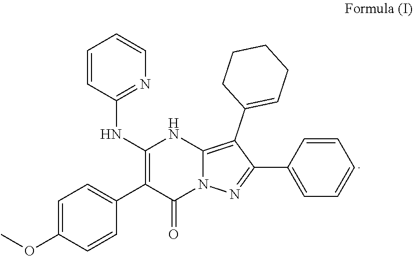 [SMILES: COc1ccc(-c2c(Nc3ccccn3)[nH]c3c(C4=CCCCC4)c(-c4ccccc4)nn3c2=O)cc1]

Aspect 65. The method of Aspect 63 or compound of Aspect 64, wherein the taxane is paclitaxel, nab-paclitaxel, or docetaxel.

Aspect 66. The method or compound of Aspect 65, wherein the taxane is docetaxel.

Aspect 67. The method or compound of Aspect 65, wherein the taxane is paclitaxel.

Aspect 68. The method of Aspect 63, further comprising administering one or more additional therapeutic agents.

Aspect 69. The compound of Aspect 64, wherein the combination further comprises one or more additional therapeutic agents.

Aspect 70. The method of Aspect 68 or compound of Aspect 69, wherein the one or more additional therapeutic agents is a platinum-based chemotherapeutic.

Aspect 71. The method or compound of Aspect 70, wherein the platinum-based chemotherapeutic is cisplatin, carboplatin, oxaliplatin, nedaplatin, triplatin tetra nitrate, phenanthriplatin, picoplatin, or satraplatin.

Aspect 72. The method or compound of Aspect 70 or 71, wherein the platinum-based chemotherapeutic is cisplatin, carboplatin, or oxaliplatin.

Aspect 73. The method of Aspect 70, further comprising administering an antimetabolite agent.

Aspect 74. The compound of Aspect 70, wherein the combination further comprises an antimetabolite agent.

Aspect 75. The method of Aspect 73 or compound of Aspect 74, wherein the antimetabolite agent is 5-fluorouracil or capecitabine.

Aspect 76. The method of Aspect 63, wherein the patient failed to respond, ceased responding, or experienced disease progression after one or more prior lines of therapy for treating MTAP-deficient esophageal cancer.

Aspect 77. The method of Aspect 63 or compound of Aspect 64, wherein the compound of Formula (I) or a pharmaceutically acceptable salt thereof is a second line of therapy for treating MTAP-deficient esophageal cancer.

Aspect 78. The method of Aspect 63 or compound of Aspect 64, wherein the compound of Formula (I) or a pharmaceutically acceptable salt thereof is a third line of therapy for treating MTAP-deficient esophageal cancer.

Aspect 79. The method of Aspect 63 or compound of Aspect 64, wherein the MTAP-deficient esophageal cancer is newly diagnosed.

Aspect 80. The method of Aspect 63 or compound of Aspect 64, wherein the dosage of the compound of Formula (I) or a pharmaceutically acceptable salt thereof is selected from once or twice daily dosing.

Aspect 81. The method of Aspect 63 or compound of Aspect 64, wherein the administration of the compound of Formula (I) or a pharmaceutically acceptable salt thereof is oral or the compound is formulated for oral administration.

Aspect 82. The method of any one of Aspects 63, 65-68, or 70-81, wherein the compound of Formula (I) or a pharmaceutically acceptable salt thereof and the taxane are administered concurrently.

Aspect 83. The method of any one of Aspects 63, 65-68, or 70-81, wherein the compound of Formula (I) or a pharmaceutically acceptable salt thereof and the taxane are administered sequentially.

Aspect 84. A method of treating a patient diagnosed with a cancer that is an MTAP-deficient lung cancer, MTAP-deficient pancreatic cancer or MTAP-deficient esophageal cancer, comprising administering:

(a) a therapeutically effective amount of a compound of Formula (I):

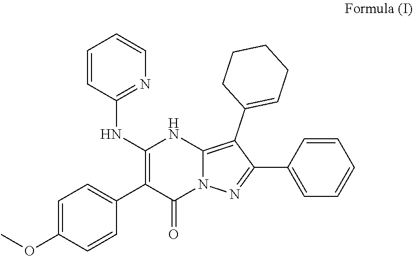 [SMILES: COc1ccc(-c2c(Nc3ccccn3)[nH]c3c(C4=CCCCC4)c(-c4ccccc4)nn3c2=O)cc1]

or a pharmaceutically acceptable salt thereof; and

(b) pemetrexed disodium.

Aspect 85. A compound of Formula (I), or a pharmaceutically acceptable salt thereof, for use in treating a cancer that is an MTAP-deficient lung cancer, MTAP-deficient pancreatic cancer or MTAP-deficient esophageal cancer, in combination with pemetrexed disodium:

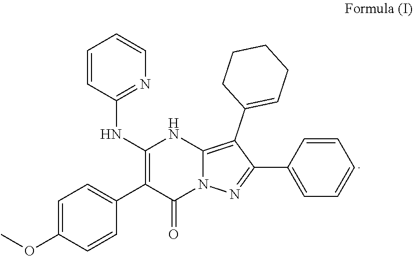 [SMILES: COc1ccc(-c2c(Nc3ccccn3)[nH]c3c(C4=CCCCC4)c(-c4ccccc4)nn3c2=O)cc1]

Aspect 86. The method of Aspect 84 or compound of Aspect 85, wherein the cancer is an MTAP-deficient lung cancer.

Aspect 87. The method or compound of Aspect 86, wherein the MTAP-deficient lung cancer is non-squamous non-small cell lung cancer.

Aspect 88. The method of any one of Aspects 84, 86, or 87, further comprising administering one or more additional therapeutic agents.

Aspect 89. The compound of any one of Aspects 85-86, wherein the combination further comprises one or more additional therapeutic agents.

Aspect 90. The method of Aspect 88 or compound of Aspect 89, wherein the additional therapeutic agent is a platinum-based chemotherapeutic.

Aspect 91. The method or compound of Aspect 90, wherein the platinum-based chemotherapeutic is cisplatin, carboplatin, oxaliplatin, nedaplatin, triplatin tetra nitrate, phenanthriplatin, picoplatin, or satraplatin.

Aspect 92. The method or compound of Aspect 90 or 91, wherein the platinum-based chemotherapeutic is carboplatin or cisplatin.

Aspect 93. The method of any of Aspects 88 or 90-92, further comprising administering pembrolizumab.

Aspect 94. The compound of any of Aspects 89-92, further comprising pembrolizumab.

Aspect 95. The method or compound of any one of Aspects 84-94 wherein the cancer is unresected, locally advanced or metastatic.

Aspect 96. A method of treating a patient diagnosed with MTAP-deficient mesothelioma, comprising administering:

(a) a therapeutically effective amount of a compound of Formula (I):

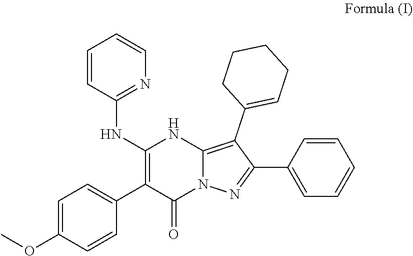 [SMILES: COc1ccc(-c2c(Nc3ccccn3)[nH]c3c(C4=CCCCC4)c(-c4ccccc4)nn3c2=O)cc1]

or a pharmaceutically acceptable salt thereof; and

(b) pemetrexed disodium.

Aspect 97. A compound of Formula (I), or a pharmaceutically acceptable salt thereof, for use in treating MTAP-deficient mesothelioma, in combination with pemetrexed disodium:

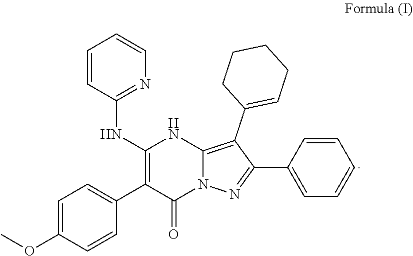 [SMILES: COc1ccc(-c2c(Nc3ccccn3)[nH]c3c(C4=CCCCC4)c(-c4ccccc4)nn3c2=O)cc1]

Aspect 98. The method of Aspect 96, further comprising administering one or more additional therapeutic agents.

Aspect 99. The compound of Aspect 97, wherein the combination further comprises one or more additional therapeutic agents.

Aspect 100. The method of Aspect 98 or compound of Aspect 99, wherein the additional therapeutic agent is a platinum-based chemotherapeutic.

Aspect 101. The method or compound of Aspect 100, wherein the platinum-based chemotherapeutic is cisplatin, carboplatin, oxaliplatin, nedaplatin, triplatin tetra nitrate, phenanthriplatin, picoplatin, or satraplatin.

Aspect 102. The method or compound of Aspect 100 or 101, wherein the platinum-based chemotherapeutic is carboplatin or cisplatin.

### Examples

The present disclosure will be more fully understood by reference to the following examples. The examples should not, however, be construed as limiting the scope of the present disclosure.

The compound of Formula (I), which, as noted above, may also be referred to as Compound 1, may be synthesized as set forth in International Application No. PCT/US2017/049439, which published as WO 2018/045071, and herein incorporated by reference in its entirety.

### Example 1: Molecule Mechanism

Cell cycle synchronization experiments were performed using a double thymidine block. These experiments were performed with HCT116 MTAP−/− (Horizon Discovery). The MTAP−/− status in this cell line was artificially engineered and was not derived from a patient sample. Double thymidine block treatment was performed to synchronize cells in early S phase after pre-treatment with the compound of Formula (I) or DMSO control for 72 hours.

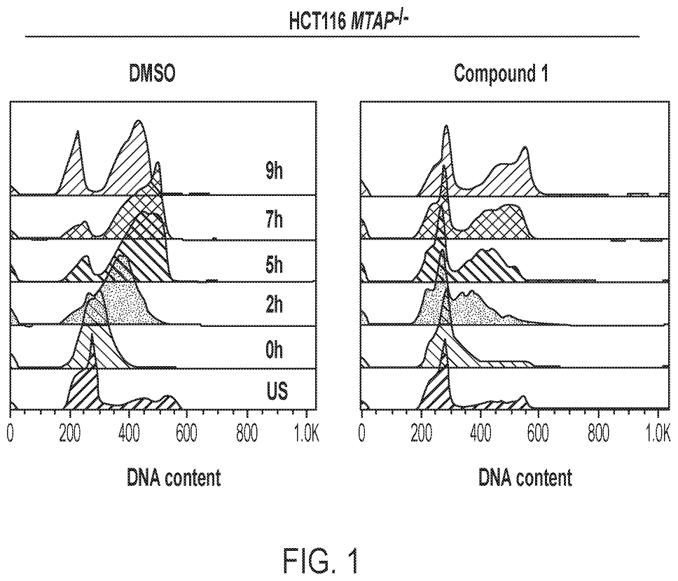

Upon subsequent release from double thymidine replicative block, cell cycle progression was monitored by flow cytometry following treatment with the compound of Formula (I) or DMSO control. Treatment with the compound of Formula (I) in HCT116 MTAP^{-/-} cells resulted in attenuated S into G2/M phase progression as evidenced by slower accumulation of cells with 4N content. FIG. 1 illustrates that the compound of Formula (I) inhibits cell cycle progression selectively in HCT116 MTAP−/− cells.

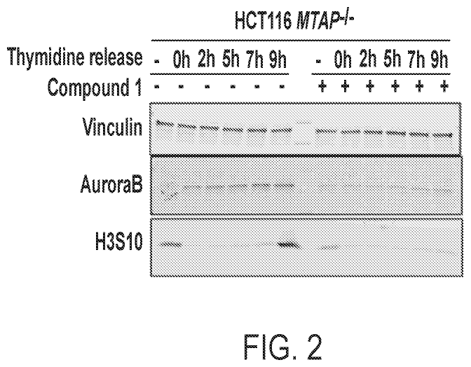

This attenuated progression through the cell cycle was further correlated with reduced protein levels of the critical mitotic regulator Aurora kinase B, as well as attenuated appearance of a mitotic marker, phosphorylated histone H3 (phS10-H3) upon release from a double thymidine block. FIG. 2 illustrates a Western blot analysis for levels of Aurora B and phospho-Ser10-H3 during cell cycle progression.

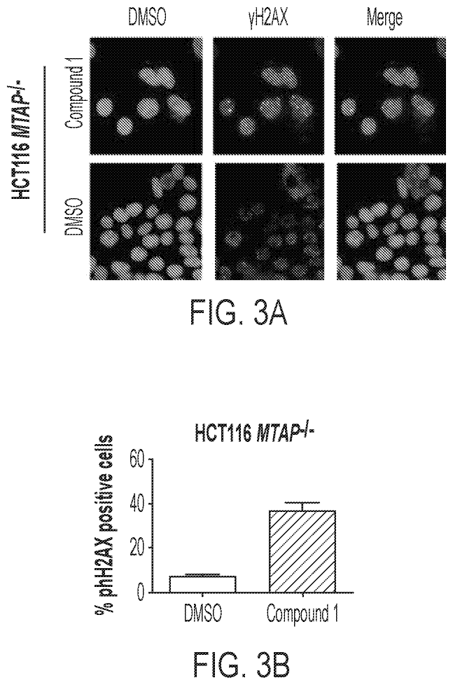

The observed reduction of the rate of progression into mitosis in HCT116 MTAP^{-/-} cells treated with Compound 1 suggests that these cells could be experiencing replication stress due to accumulation of damaged DNA. To assess the levels of DNA damage, an analysis was performed of the levels of phosphorylated H2AX (γH2AX) using immunofluorescence. The results of these experiments demonstrated more than 3-fold increase in the number of γH2AX positive cells after treatment with Compound 1 compared to the DMSO control. FIGS. 3A and 3B illustrate an immunofluorescence analysis of γH2AX, which demonstrates an increase level of DNA damage in HCT116 MTAP^{-/-} upon treatment with Compound 1.

Assessment of whether HCT116 MTAP^{-/-} cells had the ability to undergo normal cell division in the presence of Compound 1 was performed.

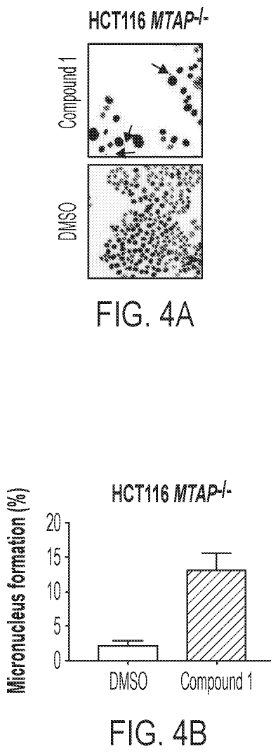

The number of cells with mitotic figure aberrations was analyzed and the frequency of micronuclei formation was assessed using DAPI staining. The results of the DAPI staining analysis demonstrated an increase in the number of micronuclei upon treatment with Compound 1. As illustrated in FIGS. 4A and 4B, a DAPI staining immunofluorescence analysis demonstrates that treatment with Compound 1 leads to increased number of micronuclei in HCT116 MTAP^{-/-} cells.

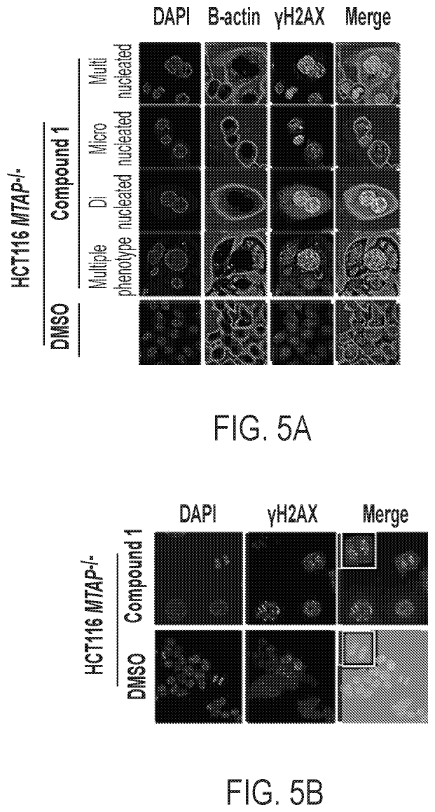

In addition, DAPI staining analysis revealed an increased number of cells with other mitotic defects associated with Compound 1 treatment in HCT116 MTAP^{-/-} cells. As illustrated in FIG. 5A, an increase in the number of cells with asymmetrically divided nuclei and di- or multinucleated cells following treatment with Compound 1.

Moreover, as illustrated in FIG. 5B, an increase in mitotic cells with chromosomal aberrations marked with γH2AX was also observed.

### Example 2: Synergy Scoring

Growth inhibition assays were performed on a panel of 29 MTAP null cell lines, shown in Table 1, including HCT116 MTAP null, utilizing Horizon Discovery's High Throughput Screening platform to assess advantageous interactions between Compound 1 and current standard of care drugs (pemetrexed, paclitaxel and gemcitabine hydrochloride. Cells were treated with 4 combinations consisting of Compound 1 in combination with five (5) agents, herein referred to as “Enhancers,” namely pemetrexed, paclitaxel, gemcitabine, ABT-348, and AZD1152-HQPA.

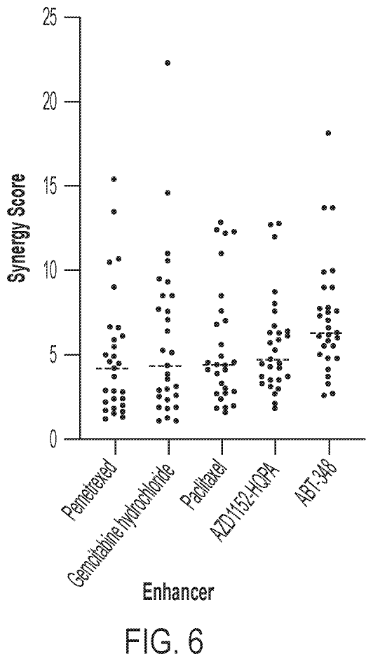

As illustrated in FIG. 6, Loewe Synergy Scores are displayed as a scatter plot and ranked by median synergy score across the cell line panel, represented by the depicted line therein.

TABLE 1
Cell Line Pattern

| | | | Observed | Assay |
|---|---|---|---|---|
| | | | Doubling | Treatment |
| Cell Line | Tissue | Media | Time (h) | Time (h) |
| HCT-116_MTAP | Colorectal | McCoy's 5A + 10% FBS | 27 | 96 |
| (−/−) | | | | |
| TE-10 | Esophagus | RPMI + 10% FBS | 39 | 96 |
| TE-14 | Esophagus | RPMI + 10% FBS | 37 | 96 |
| TE-6 | Esophagus | RPMI + 10% FBS | 36 | 96 |
| A549 | Lung | Hams F12K + 10% FBS | 31 | 96 |
| HCC1171 | Lung | RPMI + 10% FBS + 25 mM HEPES + 25 | 45 | 96 |
| | | mM sodium bicarbonate | | |
| HCC-15 | Lung | RPMI + 10% FBS | 35 | 96 |
| HLC-1 | Lung | Ham's F12 + 10% FBS | 44 | 96 |
| LU-99 | Lung | RPMI + 10% FBS | 29 | 96 |
| NCI-H1437 | Lung | RPMI + 10% FBS | 37 | 96 |
| NCI-H1650 | Lung | RPMI + 10% FBS | 39 | 96 |
| NCI-H1755 | Lung | RPMI + 10% FBS | 27 | 96 |
| NCI-H2023 | Lung | HITES + 5% FBS | 33 | 96 |
| NCI-H2126 | Lung | HITES | 51 | 96 |
| NCI-H2170 | Lung | RPMI + 10% FBS | 43 | 96 |
| NCI-H2228 | Lung | RPMI + 10% FBS | 67 | 96 |
| NCI-H647 | Lung | RPMI + 10% FBS | 34 | 96 |
| NCI-H838 | Lung | RPMI + 10% FBS | 31 | 96 |
| SK-LU-1 | Lung | EMEM + 10% FBS | 43 | 96 |
| SW1573 | Lung | RPMI + 10% FBS | 34 | 96 |
| SW900 | Lung | RPMI + 10% FBS | 49 | 96 |
| BxPC-3 | Pancreas | RPMI + 10% FBS | 36 | 96 |
| DAN-G | Pancreas | RPMI + 10% FBS | 43 | 96 |
| HuP-T3 | Pancreas | EMEM + 10% FBS + 1% NEAA + 1 mM | 38 | 96 |
| | | Sodium Pyruvate | | |
| KP-4 | Pancreas | RPMI + 10% | 28 | 96 |
| MIA PaCa-2 | Pancreas | DMEM + 10% FBS + 2.5% horse serum | 29 | 96 |
| PK-45H | Pancreas | RPMI + 10% FBS | 42 | 96 |
| SNU-410 | Pancreas | RPMI + 10% FBS + 25 mM HEPES + 25 | 60 | 96 |
| | | mM sodium bicarbonate | | |
| SU.86.86 | Pancreas | RPMI + 10% FBS | 34 | 96 |

The following tables provide the results of the Loewe Synergy Scores, the calculation of which is described hereinabove, resulting from Example 2.

TABLE 2
Combination Response - Compound 1 × ABT-384

| | | | Synergy | Loewe | |
|---|---|---|---|---|---|
| Cell Line | Tissue | Enhancer | Score | Volume | N |
| TE-10 | Esophagus | ABT-348 | 13.7 | 10.0 | 3 |
| TE-14 | Esophagus | ABT-348 | 9.0 | 9.1 | 4 |
| TE-6 | Esophagus | ABT-348 | 4.8 | 6.7 | 3 |
| A549 | Lung | ABT-348 | 5.5 | 3.3 | 3 |
| HCC1171 | Lung | ABT-348 | 7.3 | 7.4 | 4 |
| HCC-15 | Lung | ABT-348 | 9.0 | 7.3 | 3 |
| HLC-1 | Lung | ABT-348 | 5.5 | 3.3 | 2 |
| LU-99 | Lung | ABT-348 | 6.6 | 6.0 | 6 |
| NCI-H1437 | Lung | ABT-348 | 7.6 | 6.8 | 3 |
| NCI-H1650 | Lung | ABT-348 | 5.8 | 3.4 | 4 |
| NCI-H1755 | Lung | ABT-348 | 6.0 | −7.4 | 4 |
| NCI-H2023 | Lung | ABT-348 | 13.7 | 11.3 | 4 |
| NCI-H2126 | Lung | ABT-348 | 3.7 | −0.9 | 4 |
| NCI-H2170 | Lung | ABT-348 | 7.7 | 5.6 | 5 |
| NCI-H2228 | Lung | ABT-348 | 5.0 | 2.7 | 3 |
| NCI-H647 | Lung | ABT-348 | 2.7 | 0.9 | 5 |
| NCI-H838 | Lung | ABT-348 | 9.9 | 9.4 | 3 |
| SK-LU-1 | Lung | ABT-348 | 2.6 | 1.7 | 3 |
| SW1573 | Lung | ABT-348 | 4.8 | 5.1 | 3 |
| SW900 | Lung | ABT-348 | 6.1 | −0.8 | 4 |
| BxPC-3 | Pancreas | ABT-348 | 7.8 | 7.4 | 7 |
| DAN-G | Pancreas | ABT-348 | 6.2 | 5.2 | 2 |
| HuP-T3 | Pancreas | ABT-348 | 7.5 | 6.7 | 9 |
| KP-4 | Pancreas | ABT-348 | 7.1 | 5.8 | 6 |
| MIA PaCa-2 | Pancreas | ABT-348 | 6.3 | 5.7 | 2 |
| PK-45H | Pancreas | ABT-348 | 3.3 | 3.3 | 5 |
| SNU-410 | Pancreas | ABT-348 | 18.1 | 16.4 | 4 |
| SU.86.86 | Pancreas | ABT-348 | 4.1 | 3.2 | 4 |

TABLE 3
Combination Response - Compound
1 × Gemcitabine hydrochloride

| | | | Synergy | Loewe | |
|---|---|---|---|---|---|
| Cell Line | Tissue | Enhancer | Score | Volume | N |
| TE-10 | Esophagus | Gemcitabine | 22.3 | 13.0 | 3 |
| | | Hydrochloride | | | |
| TE-14 | Esophagus | Gemcitabine | 4.4 | 2.0 | 4 |
| | | Hydrochloride | | | |
| TE-6 | Esophagus | Gemcitabine | 1.9 | 2.2 | 6 |
| | | Hydrochloride | | | |
| A549 | Lung | Gemcitabine | 3.6 | 2.5 | 5 |
| | | Hydrochloride | | | |
| HCC1171 | Lung | Gemcitabine | 2.9 | 3.0 | 7 |
| | | Hydrochloride | | | |
| HCC-15 | Lung | Gemcitabine | 5.3 | 2.1 | 3 |
| | | Hydrochloride | | | |
| HLC-1 | Lung | Gemcitabine | 3.9 | −0.2 | 3 |
| | | Hydrochloride | | | |
| LU-99 | Lung | Gemcitabine | 1.1 | −2.7 | 3 |
| | | Hydrochloride | | | |
| NCI-H1437 | Lung | Gemcitabine | 7.7 | 4.6 | 2 |
| | | Hydrochloride | | | |
| NCI-H1650 | Lung | Gemcitabine | 8.5 | 4.9 | 3 |
| | | Hydrochloride | | | |
| NCI-H1755 | Lung | Gemcitabine | 2.4 | −0.8 | 6 |
| | | Hydrochloride | | | |
| NCI-H2023 | Lung | Gemcitabine | 11.0 | 8.0 | 4 |
| | | Hydrochloride | | | |
| NCI-H2126 | Lung | Gemcitabine | 1.8 | −2.8 | 4 |
| | | Hydrochloride | | | |
| NCI-H2170 | Lung | Gemcitabine | 9.3 | 3.0 | 7 |
| | | Hydrochloride | | | |
| NCI-H2228 | Lung | Gemcitabine | 5.1 | 2.6 | 5 |
| | | Hydrochloride | | | |
| NCI-H647 | Lung | Gemcitabine | 3.0 | 2.6 | 5 |
| | | Hydrochloride | | | |
| NCI-H838 | Lung | Gemcitabine | 3.1 | 2.3 | 6 |
| | | Hydrochloride | | | |
| SK-LU-1 | Lung | Gemcitabine | 8.5 | 4.5 | 4 |
| | | Hydrochloride | | | |
| SW1573 | Lung | Gemcitabine | 10.6 | 7.3 | 5 |
| | | Hydrochloride | | | |
| SW900 | Lung | Gemcitabine | 6.4 | 2.5 | 5 |
| | | Hydrochloride | | | |
| BxPC-3 | Pancreas | Gemcitabine | 14.6 | 11.5 | 3 |
| | | Hydrochloride | | | |
| DAN-G | Pancreas | Gemcitabine | 1.3 | −3.1 | 3 |
| | | Hydrochloride | | | |
| HuP-T3 | Pancreas | Gemcitabine | 7.1 | 5.2 | 8 |
| | | Hydrochloride | | | |
| KP-4 | Pancreas | Gemcitabine | 2.5 | 0.2 | 2 |
| | | Hydrochloride | | | |
| MIA PaCa-2 | Pancreas | Gemcitabine | 2.6 | −1.9 | 5 |
| | | Hydrochloride | | | |
| PK-45H | Pancreas | Gemcitabine | 1.1 | −0.2 | 3 |
| | | Hydrochloride | | | |
| SNU-410 | Pancreas | Gemcitabine | 9.5 | 5.3 | 4 |
| | | Hydrochloride | | | |
| SU.86.86 | Pancreas | Gemcitabine | 1.9 | −0.1 | 2 |
| | | Hydrochloride | | | |

TABLE 4
Combination Response - Compound 1 × Paclitaxel

| | | | Synergy | Loewe | |
|---|---|---|---|---|---|
| Cell Line | Tissue | Enhancer | Score | Volume | N |
| TE-10 | Esophagus | Paclitaxel | 4.4 | 2.1 | 3 |
| TE-14 | Esophagus | Paclitaxel | 12.9 | 8.9 | 4 |
| TE-6 | Esophagus | Paclitaxel | 2.0 | 1.8 | 3 |
| A549 | Lung | Paclitaxel | 2.7 | 4.4 | 4 |
| HCC1171 | Lung | Paclitaxel | 2.8 | 1.8 | 4 |
| HCC-15 | Lung | Paclitaxel | 6.8 | 4.4 | 3 |
| HLC-1 | Lung | Paclitaxel | 12.4 | 9.2 | 4 |
| LU-99 | Lung | Paclitaxel | 1.6 | 1.9 | 3 |
| NCI-H1437 | Lung | Paclitaxel | 4.4 | 1.4 | 3 |
| NCI-H1650 | Lung | Paclitaxel | 2.7 | 2.4 | 4 |
| NCI-H1755 | Lung | Paclitaxel | 4.6 | 2.4 | 3 |
| NCI-H2023 | Lung | Paclitaxel | 7.0 | 6.7 | 4 |
| NCI-H2126 | Lung | Paclitaxel | 1.2 | −4.9 | 4 |
| NCI-H2170 | Lung | Paclitaxel | 4.9 | 2.0 | 9 |
| NCI-H2228 | Lung | Paclitaxel | 7.6 | 3.8 | 3 |
| NCI-H647 | Lung | Paclitaxel | 12.2 | 8.8 | 6 |
| NCI-H838 | Lung | Paclitaxel | 2.4 | −1.0 | 3 |
| SK-LU-1 | Lung | Paclitaxel | 3.9 | 3.5 | 3 |
| SW1573 | Lung | Paclitaxel | 3.0 | 3.7 | 3 |
| SW900 | Lung | Paclitaxel | 1.8 | 1.1 | 5 |
| BxPC-3 | Pancreas | Paclitaxel | 3.3 | 2.5 | 8 |
| DAN-G | Pancreas | Paclitaxel | 5.6 | 5.3 | 6 |
| HuP-T3 | Pancreas | Paclitaxel | 12.3 | 8.5 | 7 |
| KP-4 | Pancreas | Paclitaxel | 8.5 | 5.6 | 5 |
| MIA PaCa-2 | Pancreas | Paclitaxel | 4.1 | 3.6 | 4 |
| PK-45H | Pancreas | Paclitaxel | 1.9 | −1.4 | 3 |
| SNU-410 | Pancreas | Paclitaxel | 11.0 | 7.8 | 4 |
| SU.86.86 | Pancreas | Paclitaxel | 4.6 | 4.6 | 3 |

TABLE 5
Combination Response - Compound 1 × AXD1152-HQPA

| | | | Synergy | Loewe | |
|---|---|---|---|---|---|
| Cell Line | Tissue | Enhancer | Score | Volume | N |
| TE-10 | Esophagus | AZD1152-HQPA | 8.7 | 7.7 | 3 |
| TE-14 | Esophagus | AZD1152-HQPA | 5.7 | 8.8 | 4 |
| TE-6 | Esophagus | AZD1152-HQPA | 3.7 | 5.8 | 4 |
| A549 | Lung | AZD1152-HQPA | 3.1 | 4.3 | 4 |
| HCC1171 | Lung | AZD1152-HQPA | 3.5 | 4.8 | 4 |
| HCC-15 | Lung | AZD1152-HQPA | 12.0 | 10.8 | 3 |
| HLC-1 | Lung | AZD1152-HQPA | 5.3 | 5.4 | 3 |
| LU-99 | Lung | AZD1152-HQPA | 4.2 | 4.5 | 3 |
| NCI-H1437 | Lung | AZD1152-HQPA | 4.5 | 5.4 | 3 |
| NCI-H1650 | Lung | AZD1152-HQPA | 3.7 | 4.1 | 4 |
| NCI-H1755 | Lung | AZD1152-HQPA | 8.0 | 0.8 | 6 |
| NCI-H2023 | Lung | AZD1152-HQPA | 6.7 | 8.1 | 4 |
| NCI-H2126 | Lung | AZD1152-HQPA | 6.3 | 5.3 | 4 |
| NCI-H2170 | Lung | AZD1152-HQPA | 12.7 | 8.9 | 5 |
| NCI-H2228 | Lung | AZD1152-HQPA | 3.5 | 5.2 | 4 |
| NCI-H647 | Lung | AZD1152-HQPA | 6.1 | 6.0 | 4 |
| NCI-H838 | Lung | AZD1152-HQPA | 4.5 | 5.6 | 3 |
| SK-LU-1 | Lung | AZD1152-HQPA | 6.4 | 7.5 | 3 |
| SW1573 | Lung | AZD1152-HQPA | 3.0 | 4.2 | 5 |
| SW900 | Lung | AZD1152-HQPA | 2.1 | 0.6 | 5 |
| BxPC-3 | Pancreas | AZD1152-HQPA | 6.3 | 8.5 | 7 |
| DAN-G | Pancreas | AZD1152-HQPA | 4.3 | 4.9 | 4 |
| HuP-T3 | Pancreas | AZD1152-HQPA | 5.9 | 5.9 | 8 |
| KP-4 | Pancreas | AZD1152-HQPA | 7.6 | 6.4 | 5 |
| MIA PaCa-2 | Pancreas | AZD1152-HQPA | 4.7 | 3.6 | 3 |
| PK-45H | Pancreas | AZD1152-HQPA | 2.7 | 4.4 | 3 |
| SNU-410 | Pancreas | AZD1152-HQPA | 12.8 | 14.0 | 4 |
| SU.86.86 | Pancreas | AZD1152-HQPA | 1.8 | 3.1 | 6 |

TABLE 6
Combination Response - Compound 1 x Pemetrexed

| | | | Synergy | Loewe | |
|---|---|---|---|---|---|
| Cell Line | Tissue | Enhancer | Score | Volume | N |
| TE-10 | Esophagus | Pemetrexed | 4.5 | 6.5 | 3 |
| TE-14 | Esophagus | Pemetrexed | 10.7 | 5.4 | 4 |
| TE-6 | Esophagus | Pemetrexed | 6.6 | 10.7 | 5 |
| A549 | Lung | Pemetrexed | 1.7 | 2.1 | 4 |
| HCC1171 | Lung | Pemetrexed | 1.2 | 0.9 | 4 |
| HCC-15 | Lung | Pemetrexed | 6.7 | 7.7 | 3 |
| HLC-1 | Lung | Pemetrexed | 2.9 | 4.0 | 3 |
| LU-99 | Lung | Pemetrexed | 1.8 | 1.2 | 3 |
| NCI-H1437 | Lung | Pemetrexed | 5.0 | 4.5 | 3 |
| NCI-H1650 | Lung | Pemetrexed | 2.8 | 5.8 | 9 |
| NCI-H1755 | Lung | Pemetrexed | 9.0 | 8.8 | 3 |
| NCI-H2023 | Lung | Pemetrexed | 4.6 | 6.1 | 4 |
| NCI-H2126 | Lung | Pemetrexed | 1.6 | 2.0 | 4 |
| NCI-H2170 | Lung | Pemetrexed | 2.2 | 0.4 | 6 |
| NCI-H2228 | Lung | Pemetrexed | 4.9 | 4.3 | 3 |
| NCI-H647 | Lung | Pemetrexed | 2.8 | 2.7 | 6 |
| NCI-H838 | Lung | Pemetrexed | 13.5 | 12.5 | 3 |
| SK-LU-1 | Lung | Pemetrexed | 5.9 | 6.6 | 7 |
| SW1573 | Lung | Pemetrexed | 1.3 | 1.9 | 4 |
| SW900 | Lung | Pemetrexed | 2.0 | 3.9 | 4 |
| BxPC-3 | Pancreas | Pemetrexed | 5.5 | 6.7 | 5 |
| DAN-G | Pancreas | Pemetrexed | 2.4 | 3.1 | 4 |
| HuP-T3 | Pancreas | Pemetrexed | 3.7 | 4.6 | 10 |
| KP-4 | Pancreas | Pemetrexed | 4.2 | 3.9 | 5 |
| MIA PaCa-2 | Pancreas | Pemetrexed | 15.4 | 10.5 | 4 |
| PK-45H | Pancreas | Pemetrexed | 1.5 | 1.3 | 3 |
| SNU-410 | Pancreas | Pemetrexed | 10.5 | 12.4 | 4 |
| SU.86.86 | Pancreas | Pemetrexed | 6.1 | 5.8 | 3 |

As demonstrated by Loewe Synergy Scores, an advantage was observed in vitro with a combination of the compound of Formula (I) in multiple MTAP null cell lines of various tumor types. Advantageous effects were observed in vitro with a combination of the compound of Formula (I) and pemetrexed, paclitaxel, gemcitabine, or Aurora kinase inhibitors (ABT-348 and AZD1152-HQPA).

### Example 3: Combination Index Assessment

Confirmatory in vitro studies were conducted with two clinically relevant chemotherapeutical agents that stabilize microtubules during mitosis, paclitaxel and docetaxel. The primary mode of action for each of paclitaxel and docetaxel is hyperstabilization of microtubules. Microtubules are composed of repeating α-tubulin and β-tubulin cytoskeletal proteins responsible for variety of cellular processes including the proper separation of chromosomes during mitosis. Paclitaxel and docetaxel directly interact with microtubules and act against microtubule de-polymerization that prevent chromosome separation as a result of the kinetochores that do not have stable attachment to microtubules. Actively dividing cancer cells treated with paclitaxel and docetaxel activate spindle assembly checkpoint leading to growth arrest in metaphase or mitotic slippage producing tetraploid cells that eventually undergo cell death. Reference is made to Montero, A., Fossella, F., Hortobagyi, G. & Valero, V. Docetaxel for treatment of solid tumours: a systematic review of clinical data. Lancet. Oncol. 6, 229-39 (2005); and Weaver, B. A. How Taxol/paclitaxel kills cancer cells. Mol. Biol. Cell 25, 2677-81 (2014).

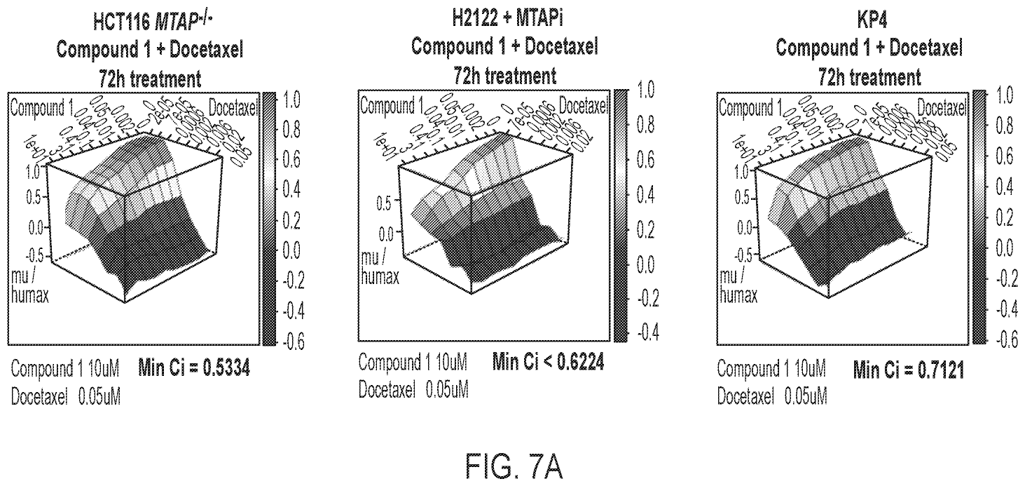

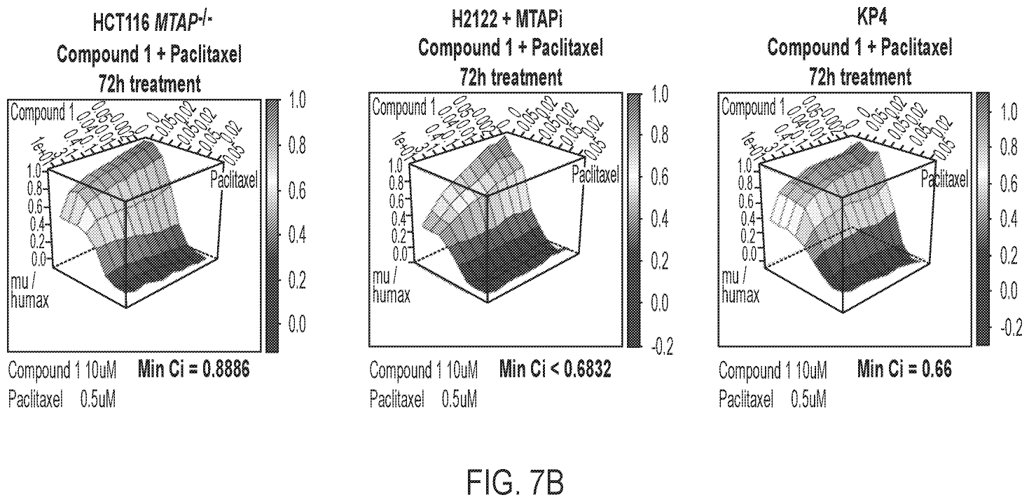

Cell growth assessment was performed using a Cell Titer-Glo assay as a readout in HCT116 MTAP−/− cell line, as well as in KP4 pancreatic MTAP−/− cell line, and H2122 MTAP wt Non-Small Lung Cancer cell line that was converted into MTAP “pharmacologic” null using an MTAP inhibitor to assess the compound of Formula I interaction with paclitaxel and docetaxel. The advantageous effects between docetaxel, paclitaxel, and the compound of Formula (I) were measured using a drug combination index (CI), described hereinabove, that gives a quantitative measure for drug combination effect. The results of the combination index assessment in HCT116 MTAP−/−, KP4, and H2122 MTAP “pharmacologic” null cells demonstrated that both paclitaxel and docetaxel have a synergistic effect on cell growth inhibition when combined with the compound of Formula (I), as illustrated in FIG. 7.

The methods and materials for Example 3 are provided hereinbelow. Tables 7-10 provide the materials for the cell growth assessment.

TABLE 7
Base media

| Item | Supplier | Cat. no. |
|---|---|---|
| RPMI | ThermoFisher (Gibco) | 21875-091 |
| DMEM | ThermoFisher (Gibco) | 41966-029 |
| F12 (Ham's F12) | ThermoFisher (Gibco) | 21765-029 |
| McCoy's 5A | ThermoFisher (Gibco) | 26600-023 |
| DMEM:F12 | ThermoFisher (Gibco) | 11320-074 |
| MEM (EMEM) | ThermoFisher (Gibco) | 31095-029 |
| Ham's F10 | ThermoFisher (Gibco) | 31550-023 |
| F12K | ThermoFisher (Gibco) | 21127-022 |

TABLE 8
Special media composition

HITES
DMEM:F12
0.005 mg/ml human insulin
0.01 mg/ml transferrin
20 nM sodium selenite
10 nM hydrocortisone
10 nM beta-estradiol
additional 2 mM L-glutamine

TABLE 9
Media supplements

| Item | Supplier | Cat. no. |
|---|---|---|
| FBS | ThermoFisher (Gibco) | 10270106 |
| Penicillin-Streptomycin | ThermoFisher (Gibco) | 15140122 |
| HEPES | ThermoFisher (Gibco) | 15630056 |
| L-glutamine | ThermoFisher (Gibco) | 25030081 |
| NEAA | ThermoFisher (Gibco) | 11140035 |
| Horse Serum | ThermoFisher (Gibco) | 16050122 |
| Sodium bicarbonate | Sigma | S5761 |
| Sodium pyruvate | Sigma | P5280 |
| Hydrocortisone | Sigma | H0888 |
| EGF | Sigma | E9644 |

TABLE 10
Other reagents

| Item | Supplier | Cat. no. |
|---|---|---|
| Trypsin | ThermoFisher (Gibco) | 25200056 |
| PBS | ThermoFisher (Gibco) | 14190169 |
| DMSO | Sigma | D2650 |
| CellTiter-Glo 2.0 | Promega | G9243 |

The method by which the High Throughput Screen was performed is herein described. The endpoint readout of this assay is based upon quantitation of ATP as an indicator of viable cells.

Cell lines that have been preserved in liquid nitrogen are thawed and expanded in growth media. Once cells have reached expected doubling times, screening begins. Cells are seeded in growth media in black 384-well tissue culture treated plates at 500-1500 cells per well (as noted in Analyzer). Cells are equilibrated in assay plates via centrifugation and placed in incubators (attached to the Dosing Modules) at 37° C. for twenty-four hours before treatment. At the time of treatment, a set of assay plates (which do not receive treatment) are collected and ATP levels are measured by adding CellTiter-Glo 2.0 (Promega). These Tzero (T0) plates are read using ultra-sensitive luminescence on Envision plate readers (Perkin Elmer). Assay plates are incubated with compound for 96 hours and are then analysed using CellTiter-Glo 2.0. All data points are collected via automated processes and are subject to quality control and analysed using Horizon's proprietary software. Assay plates are accepted if they pass the following quality control standards: relative raw values are consistent throughout the entire experiment, Z-factor scores are greater than 0.6 and untreated/vehicle controls behave consistently on the plate.

Growth Inhibition (GI) was used as a measure of cell growth. The GI percentages are calculated by applying the following test and equation:

$$If T < {V}_{0} : 100 * ( 1 - \frac{T - {V}_{0}}{{V}_{0}} )$$

$$If T \geq {V}_{0} : 100 * ( 1 - ( \frac{T - {V}_{0}}{V - {V}_{0}} )$$

where T is the signal measure for a test article, V is the untreated/vehicle-treated control measure, and Vo is the untreated/vehicle control measure at time zero (also colloquially referred as T0 plates). This formula is derived from the Growth Inhibition calculation used in the National Cancer Institute's NCI-60 high throughput screen. All data analysis was performed in Growth Inhibition (except where noted).

A GI reading of 0% represents no growth inhibition and would occur in instances where the T reading at 96 hours is comparable to the V reading at the respective time period. A GI of 100% represents complete growth inhibition (cytostasis) and in this case cells treated with compound for 96 hours would have the same endpoint reading as T0 control cells. A GI of 200% represents complete death (cytotoxicity) of all cells in the culture well and in this case the T reading at 96 hours will be lower than the T0 control (values near or at zero).

Inhibition is provided as a measure of cell viability. Inhibition levels of 0% represent no inhibition of cell growth by treatment. Inhibition of 100% represents no doubling of cell numbers during the treatment window. Both cytostatic and cytotoxic treatments can yield an Inhibition percentage of 100%. Inhibition percentage is calculated as the following: I=1−T/U, where T is the treated and U is the untreated/vehicle control.

### Example 4: Combination of the Compound of Formula (I) and Docetaxel Therapy in Pancreatic KP4 Xenograft Model

Study Objective: The objective of this study was to evaluate the potential efficacy of the compound of Formula (I) given once daily (PO), alone and in combination with Docetaxel, against established MTAP-deficient pancreatic xenograft tumors (KP4), in female mice.

Study Design: 5-6 weeks old female, CB-17 SCID mice were subcutaneously inoculated with 1×10⁷ KP4 cells in serum free media+Matrigel (1:1). The mice were randomized on Day 26 once tumors averaged 200 mm³, into treatment groups and dosed as outlined in Table 11.

TABLE 11
Study Design/Treatment Schedules

| | # of | | | |
|---|---|---|---|---|
| Group | Animals | Treatment | Route | Dose and Schedule |
| 1 | 12 | Vehicle#1: IV Q7D | IV | 5 mL/kg, (Q7D × 4) |
| | | 0.9% NaCl + | | |
| | | Vehicle#2: PO QD | PO | 10 mL/kg, (QD × 29) |
| 2 | 12 | the compound of | PO | 100 mg/kg, (QD × 29) |
| | | Formula (I) | | |
| 3 | 12 | Docetaxel | IV | 5 mg/kg, (Q7D × 4) |
| 4 | 12 | the compound of | PO + | 100 mg/kg, (QD × 29) |
| | | Formula (I) + | IV | 5 mg/Kg, (Q7D × 4) |
| | | Docetaxel | | |
| | | (combination) | | |

Materials and Methods: Tumor volumes were measured twice weekly in two dimensions using a caliper, and the volume was expressed in mm3 using the formula: V=(L×W×W)/2, where V is tumor volume, L is tumor length (the longest tumor dimension) and W is tumor width (perpendicular to L). Body weights were measured twice per week.

The compound of Formula (I) was supplied as a formulation comprising amorphous Formula (I). The compound was stored at 4° C. protected from light. The compound of Formula (I) was formulated daily in a vehicle. Formulated, the compound of Formula (I) is stable for 24 hours when stored at 4° C. protected from light.

The compound of Formula (I) was dosed orally at 100 mg/kg, daily, for Groups 2 and 4.

Docetaxel was purchased from Myoderm (Cat. No. 66758-0050-01) and formulated in 0.9% NaCl for sterile injection. Docetaxel was dosed IV using 5 mg/kg for groups 3 and 4.

Vehicle preparation for Group 1 (vehicle only) matched that of the compound of Formula (I) formulation. Vehicle was formulated fresh daily and is stable for 24 hours when stored at 4° C.

### Results:

Treatment with the vehicle was well tolerated with 0 body weight loss (BWL) during the study. Tumor volume reach a median of 1687.4 mm³ on day 19 of treatment (Table 28), Group 1 termination.

Treatment with 100 mg/kg the compound of Formula (I) alone (Group 2) was well tolerated with maximal median BWL of 3% on day 4 of treatment. Tumor volume reached a median of 1426.7 mm³ on day 36 of treatment, Group 2 termination.

Treatment with 5 mg/kg Docetaxel alone was well tolerated with a maximal median BWL of 3% on day 1 of treatment. Tumor volume reached a median of 1365.8 mm³ on day 36 of treatment, Group 3 termination.

Treatment with the combination of the compound of Formula (I) and Docetaxel was well tolerated with a median BWL of 3% on day 4 of treatment. Tumor volume reached a median of 1075.3 mm³ on day 54 of treatment, Group 4 termination.

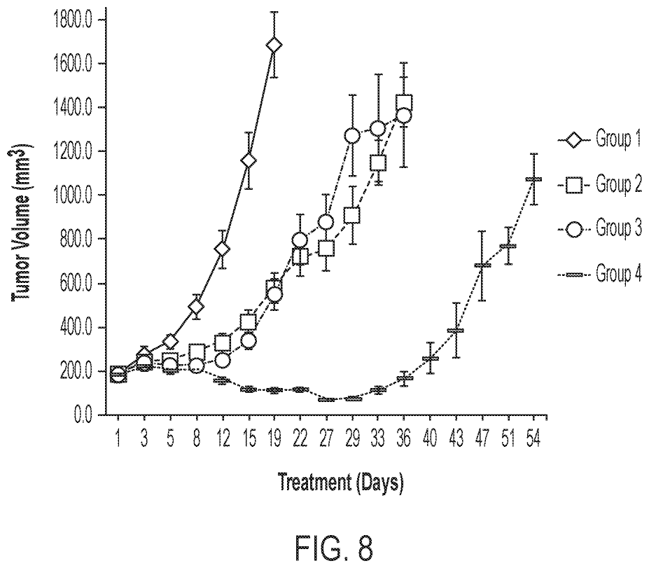

The tumor volumes from each group are shown in Table 12 and are illustrated graphically in FIG. 8. The combination methodology is described herein and the results are shown in Table 13.

TABLE 12
Measured Tumor Volumes and SEM

| Day (post | Group 1 | | Group 2 | | Group 3 | | Group 4 | |
|---|---|---|---|---|---|---|---|---|
| treatment start) | Median (SEM) | | Median (SEM) | | Median (SEM) | | Median (SEM) | |
| 1 | 184.6 | (15.9) | 186.1 | (17.0) | 189.4 | (16.8) | 187.0 | (16.1) |
| 3 | 281.6 | (30.1) | 245.6 | (18.7) | 236.6 | (20.9) | 224.6 | (23.0) |
| 5 | 335.2 | (29.2) | 251.2 | (22.3) | 224.0 | (16.8) | 205.4 | (17.3) |
| 8 | 494.3 | (55.9) | 288.1 | (27.6) | 229.2 | (21.3) | 213.6 | (17.6) |
| 12 | 755.7 | (84.0) | 330.8 | (42.5) | 257.5 | (24.6) | 158.7 | (14.7) |
| 15 | 1159.8 | (128.9) | 426.1 | (52.4) | 339.1 | (37.3) | 117.8 | (13.1) |
| 19 | 1687.4 | (147.3) | 582.8 | (68.9) | 550.0 | (69.2) | 113.0 | (12.2) |
| 22 | | | 724.9 | (91.3) | 798.2 | (113.3) | 115.9 | (10.5) |
| 26 | | | 557.7 | (59.1) | 867.8 | (135.0) | 65.1 | (8.1) |
| 27 | | | 759.8 | (99.9) | 877.8 | (127.3) | 72.6 | (8.0) |
| 29 | | | 911.1 | (133.6) | 1273.6 | (182.3) | 78.0 | (9.2) |
| 33 | | | 1150.9 | (100.9) | 1307.6 | (241.5) | 113.4 | (17.6) |
| 36 | | | 1426.7 | (113.5) | 1365.8 | (236.3) | 166.6 | (33.1) |
| 40 | | | | | | | 259.3 | (68.4) |
| 43 | | | | | | | 385.8 | (125.7) |
| 47 | | | | | | | 681.2 | (158.1) |
| 51 | | | | | | | 773.4 | (82.9) |
| 54 | | | | | | | 1075.3 | (112.6) |

TABLE 13
Combination Statistical Analysis for days 0-19

Mean AUC Group 1 = 17.673327
Mean AUC Group 2 = 9.078992
Mean AUC Group 3 = 8.951130
Mean AUC Group 4 = −7.824595
In vivo Synergy Score : −46.2923059814358
p value : 0.000255197802850549

### Example 5: Combination of the Compound of Formula (I) and Paclitaxel Therapy in a Pancreatic Cancer Xenograft Model (PA0372) in Female BALB/c Nude Mice

Study Objective: The objective of this study was to evaluate the potential of the therapeutic efficacy of the compound of Formula (I) as single treatment or combination treatment with Paclitaxel of HuPrime® pancreatic xenograft model PA0372 (an MTAP-deficient model) in female BALB/c nude mice.

Study Design: PA0372 Tumor fragments were inoculated into BALB/c nude mice and treatment was initiated when tumors reached mean tumor volume around 158 mm³. The test agent the compound of Formula (I) as single agent at 100 mg/kg (group 2), and combined with 15 mg/kg Paclitaxel.

TABLE 14
Study Design/Treatment Schedules

| | # of | | | |
|---|---|---|---|---|
| Group | Animals | Treatment | Route | Dose and Schedule |
| 1 | 12 | Vehicle | PO | QD × 29 days |
| 2 | 12 | The compound | PO | 100 mg/kg QD × 29 |
| | | of Formula (I) | | days |
| 3 | 12 | Paclitaxel | IP | 15 mg/kg days 1,8, 15, 29 |
| 4 | 12 | The compound | PO | 100 mg/kg QD × 29 days |
| | | of Formula (I) | IP | 15 g/kg days 1, 8, 15, |
| | | Paclitaxel | | 29 |

Materials and Methods: Tumor volumes were measured twice weekly in two dimensions using a caliper, and the volume was expressed in mm³ using the formula: V=(L×W×W)/2, where V is tumor volume, L is tumor length (the longest tumor dimension) and W is tumor width (perpendicular to L). Body weights were measured twice per week.

The compound of Formula (I) was supplied as a formulation comprising amorphous Formula (I). The compound was stored at 4° C. protected from light. The compound of Formula (I) was formulated daily in a vehicle. Formulated, the compound of Formula (I) is stable for 24 hours when stored at 4° C. protected from light.

The compound of Formula (I) was dosed orally at 100 mg/kg, daily, for Groups 2 and 4.

Paclitaxel was purchased from Selleck (Cat. No. S1150) and formulated in 5% DMSO+5% Tween 80+90% ddH2O. Paclitaxel was dosed IV using 15 mg/kg for groups 3 and 4.

Vehicle preparation for Group 1 (vehicle only) matched that of the compound of Formula (I) formulation. Vehicle was formulated fresh daily and is stable for 24 hours when stored at 4° C.

Tumors from stock mice bearing PA0372 human primary pancreatic tumors were harvested, dissected into fragments and inoculated into BALB/c nude mice.

Each mouse was inoculated subcutaneously in the right flank with PA0372 fragment (P5, 2-4 mm in diameter) for tumor development.

Tumored animals were randomly allocated to the 4 different study groups, based on their tumor volume. The mean tumor volume at randomization was 158 mm³. The day of randomization and dosing initiation was defined as study Day 1.

### Results:

Treatment with the vehicle was well tolerated with 0 body weight loss (BWL) during the study. Tumor volume reached a median of 1220.39 mm³ on day 29 of treatment, Group 1 termination.

Treatment with the 100 mg/kg the compound of Formula (I) was well tolerated with 0 body weight loss (BWL) during the study. Tumor volume reached a median of 596.13 mm³ on day 29 of treatment, Group 2 termination.

Treatment with the 15 mg/kg Paclitaxel was well tolerated with 0 body weight loss (BWL) during the study. Tumor volume reached a median of 913.79 mm³ on day 29 of treatment, Group 3 termination.

Treatment with 100 mg/kg the compound of Formula (I) combined with 15 mg/kg Paclitaxel was well tolerated with 0 body weight loss (BWL) during the study. Tumor volume reached a median of 326.71 mm³ on day 29 of treatment, Group 4 termination.

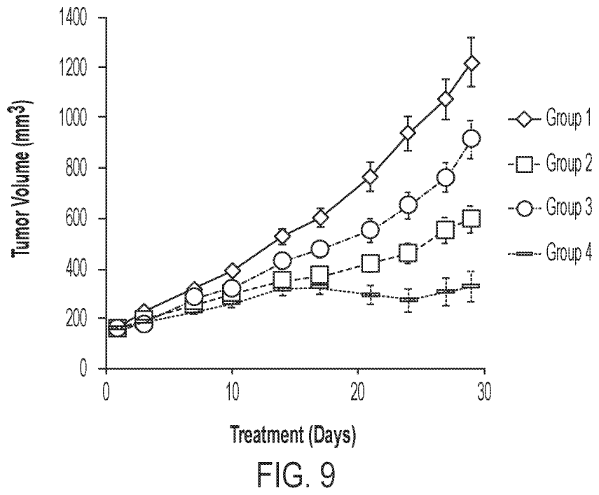

The results of Table 15 are illustrated in FIG. 9. The combination methodology is described herein and the results are shown in Table 16.

TABLE 15
Measured Tumor Volumes and SEM

| Day (post | Group 1 | | Group 2 | | Group 3 | | Group 4 | |
|---|---|---|---|---|---|---|---|---|
| treatment start) | Median (SEM) | | Median (SEM) | | Median (SEM) | | Median (SEM) | |
| 1 | 158.24 | (9.09) | 158.09 | (8.96) | 158.15 | (8.96) | 157.97 | (8.86) |
| 3 | 224.88 | (13.00) | 194.53 | (13.28) | 181.36 | (9.46) | 185.23 | (8.01) |
| 7 | 317.60 | (17.55) | 255.54 | (17.84) | 280.14 | (16.52) | 231.89 | (14.36) |
| 10 | 388.12 | (19.29) | 296.37 | (17.62) | 320.03 | (18.49) | 261.43 | (18.51) |
| 14 | 527.92 | (31.01) | 350.19 | (22.39) | 428.65 | (30.29) | 317.17 | (27.17) |
| 17 | 602.13 | (36.88) | 371.89 | (22.98) | 475.71 | (34.16) | 324.04 | (28.01) |
| 21 | 763.43 | (56.42) | 417.05 | (33.40) | 551.46 | (49.35) | 292.00 | (37.65) |
| 24 | 937.64 | (71.76) | 458.16 | (38.02) | 650.46 | (52.51) | 275.91 | (45.87) |
| 27 | 1073.57 | (80.67) | 551.89 | (50.41) | 760.49 | (58.91) | 306.61 | (55.65) |
| 29 | 1220.33 | (94.41) | 596.13 | (53.32) | 913.79 | (75.84) | 326.71 | (62.36) |

TABLE 16
Combination Statistical Analysis for days 0-29

Mean AUC Group 1 = 14.294180
Mean AUC Group 2 = 9.102725
Mean AUC Group 3 = 11.309317
Mean AUC Group 4 = 5.852445
In vivo Synergy Score : −1.85681530064591
p value : 0.827797042135567

### Example 6: Combination of the Compound of Formula (I) and Paclitaxel Therapy in Pancreatic PAX41 POX Model

Study Objective: The objective of this study was to evaluate the efficacy of the compound of Formula (I), given once daily (P0) alone and in combination with Paclitaxel, against an established patient derived MTAP-deficient xenograft tumors (POX), PAX041, in female Nu/Nu mice.

Study Design: The study mice were randomized on Day 23 post inoculation into four study groups based on a median tumor volume of 133 mm³. Treatment began on Day 23 post inoculation (first day of treatment denoted as day 1) with the treatment schedules summarized in Table 17.

TABLE 17
Study Design/Treatment Schedules

| | # of | | | |
|---|---|---|---|---|
| Group | Animals | Treatment | Route | Dose and Schedule |
| 1 | 12 | Vehicle Control | PO | QD × 28 days |
| 2 | 12 | the compound of | PO | 100 mpk QD × 28 days |
| | | Formula (I) | | |
| 3 | 12 | Paclitaxel | IP | 7.5 mg/kg days 1, 8, 22, 28 |
| | | the compound of | PO + | 100 mg/kg QD × 28 days |
| 4 | 12 | Formula (I) + | IP | 7.5 mg/kg days 1, 8, 22, 28 |
| | | Paclitaxel | | |
| | | (simultaneous | | |
| | | treatment) | | |

Materials and Methods: Female Nu/Nu mice weighing 18-22 g were purchased from Beijing Vital River Laboratory Animal Technology Co. Ltd. (Beijing, China) and subcutaneously implanted with PAX041 tumor fragments. PAX041 is a human MTAP-deficient primary pancreatic cancer xenograft model established at ChemPartner. Treatment began on Day 23 with the dosing schedules set forth in Table 17.

Tumor volumes were measured twice weekly in two dimensions using a caliper, and the volume was expressed in mm³ using the formula: V=(L×W×W)/2, where V is tumor volume, L is tumor length (the longest tumor dimension) and W is tumor width (perpendicular to L). Tumor growth inhibition rate (TGI %) of each dosing group was calculated according to the following formula: TGI %=[1−(TVi−TV0)/(TVvi−TVv0)]×100%; TVi is average tumor volume of a dosing group on a specific day; TV0 is average tumor volume of a dosing group on the initial day; TVvi is average tumor volume of the vehicle group on a specific day; TVv0 is average tumor volume of the vehicle group on the initial day. Body weights were measured twice per week.

The compound of Formula (I) was supplied as a formulation comprising amorphous Formula (I). The compound was stored at 4° C. protected from light. The compound of Formula (I) was formulated daily in a vehicle. Formulated, the compound of Formula (I) is stable for 24 hours when stored at 4° C. protected from light.

The compound of Formula (I) was dosed orally at 100 mg/kg, daily, for Groups 2 and 4.

Paclitaxel was purchased from Selleck in China (Cat. No. S1150) and formulated in 5% DMSO, 5% Tween 80 and ddH2O. Paclitaxel was dosed IP using 7.5 mpk for Groups 3 and 4.

Vehicle preparation for Group 1 (vehicle only) matched that of the compound of Formula (I) formulation. Vehicle was formulated fresh daily and is stable for 24 hours when stored at 4° C.

### Results:

Treatment with the vehicle (Group 1) was well tolerated with 0 body weight loss (BWL) during the study. Tumor volume reach a median of 847 mm³ on day 28, study termination.

Treatment with 100 mg/kg the compound of Formula (I) alone (Group 2) was well tolerated with maximal median BWL of 3% on day 12 of treatment. Tumor volume reach a median of 461 mm³ on day 28 (TGI=55%), study termination.

Treatment with 7.5 mg/kg Paclitaxel alone (Group 3) was well tolerated with a maximal median BWL of 1% on day 2. Tumor volume reach a median of 756 mm³ on day 28 (TGI=12%), study termination. It should be noted a higher dose of Paclitaxel (15 mg/kg) dosed IP on days 1, 8, and 22 was explored in this study. This dose was not tolerated as 2 of 12 animals found dead on day 20 and 22, respectively.

Treatment with the combination of the compound of Formula (I) and Paclitaxel (Group 4) was well tolerated with a median BWL of 4% on day 11. Tumor volume reach a median of 491 mm³ on day 28 (TGI=50%), study termination. It should be noted a higher dose of Paclitaxel (15 mg/kg) dosed IP on days 1, 8 combined with the compound of Formula (I) (100 mg/kg) was explored in this study. This combination was not tolerated as 3 of 12 animals found dead on day 20.

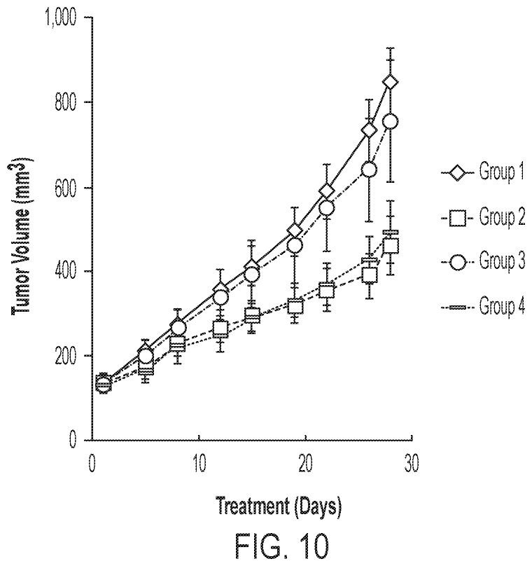

The results of Table 18 are illustrated in FIG. 10. The combination methodology is described herein and the results are shown in Table 19.

TABLE 18
Measured Tumor Volumes and SEM

| Day (post | Group 1 | | Group 2 | | Group 3 | | Group 4 | |
|---|---|---|---|---|---|---|---|---|
| treatment start) | Median (SEM) | | Median (SEM) | | Median (SEM) | | Median (SEM) | |
| 1 | 213.56 | (17.55) | 136.46 | (20.36) | 131.21 | (17.42) | 131.02 | (16.48) |
| 5 | 213.56 | (25.22) | 171.99 | (26.20) | 200.27 | (34.46) | 164.83 | (27.72) |
| 8 | 274.53 | (36.44) | 229.40 | (29.53) | 263.46 | (44.22) | 222.67 | (42.03) |
| 12 | 355.95 | (48.03) | 264.21 | (32.19) | 338.84 | (64.70) | 247.50 | (38.44) |
| 15 | 412.91 | (47.07) | 294 | (35.31) | 392.76 | (78.34) | 289.60 | (36.21) |
| 19 | 493.47 | (58.19) | 319.51 | (42.90) | 462.46 | (88.86) | 331.85 | (39.38) |
| 22 | 588.02 | (64.77) | 356.12 | (50.74) | 550.87 | (102.54) | 369.03 | (48.28) |
| 26 | 733.22 | (73.05) | 389.77 | (53.38) | 639.8 | (123.19) | 427.41 | (55.99) |
| 28 | 847.34 | (79.74) | 460.86 | (68.22) | 756.49 | (143.62) | 491.58 | (72.81) |

TABLE 19
Combination Statistical Analysis for days 0-28

Mean AUC Group 1 = 10.694597
Mean AUC Group 2 = 7.152290
Mean AUC Group 3 = 8.770094
Mean AUC Group 4 = 7.068225
In vivo Synergy Score: 17.2090546384413
p-value: 0.286373843001645

### Example 7. Combination of the Compound Shown in Formula (I) and Docetaxel Therapy in Esophageal ESX030 PDX Model

Study Objective: The objective of this study was to evaluate the efficacy of the compound shown in Formula (I), given once daily (PO) alone and in combination with Docetaxel, against an established patient derived xenograft tumors (PDX), ESX030, in female Nu/Nu mice.

Study Design:

The study mice were randomized on Day 20 post inoculation into four study groups based on a median tumor volume of 142 mm³. Treatment began on Day 20 post inoculation (first day of treatment denoted as day 1) with the treatment schedules summarized in Table 20.

TABLE 20
Study Design/Treatment Schedules

| | # of | | | |
|---|---|---|---|---|
| Group | Animals | Treatment | Route | Dose and Schedule |
| 1 | 12 | Vehicle Control | PO | QD × 36 days |
| 2 | 12 | Compound of | PO | 100 mpk QD × 57 days |
| | | Formula I | | |
| 3 | 12 | Docetaxel | IV | 2.5 mg/kg Q7D |
| 4 | 12 | Docetaxel | IV | 5.0 mg/kg Q14D for 4 |
| | | | | weeks then Q7D |
| 5 | 12 | Compound of | PO + | 100 mg/kg QD × 71 days |
| | | Formula (I) + | IV | 2.5 mg/kg Q7D |
| | | Docetaxel | | |
| | | (simultaneous | | |
| | | treatment) | | |
| 6 | 12 | Compound of | PO + | 100 mg/kg QD × 71 days |
| | | Formula (I) + | IV | 5.0 mg/kg Q14D for 4 |
| | | Docetaxel | | weeks then Q7D |
| | | (simultaneous | | |
| | | treatment) | | |

Material and Methods:

Female Nu/Nu mice weighing 18-22 g were purchased from Beijing Vital River Laboratory Animal Technology Co. Ltd. (Beijing, China) and subcutaneously implanted with ESX030 tumor fragments. ESX030 is a human primary esophageal cancer xenograft model established at ChemPartner.

Tumor volumes were measured twice weekly in two dimensions using a caliper, and the volume was expressed in mm³ using the formula: V=(L×W×W)/2, where V is tumor volume, L is tumor length (the longest tumor dimension) and W is tumor width (perpendicular to L). Tumor growth inhibition rate (TGI %) of each dosing group was calculated according to the following formula: TGI %=[1−(TVᵢ−TV₀)/(TVᵥᵢ−TVᵥ₀)]×100%; TVᵢ is average tumor volume of a dosing group on a specific day; TV₀ is average tumor volume of a dosing group on the initial day; TVᵥᵢ is average tumor volume of the vehicle group on a specific day; TVᵥ₀ is average tumor volume of the vehicle group on the initial day.

Body weights were measured twice per week.

Compound shown in Formula (I) was supplied as a formulation containing 25% active pharmaceutical ingredient (API). The compound was stored at 4° C. protected from light. The compound of Formula (I) was formulated daily in a vehicle. Formulated, the compound of Formula (I) is stable for 24 hours when stored at 4° C. protected from light.

The compound of Formula I was dosed orally at 100 mg/kg, daily, for Groups 2, 5 and 6. The dose of the compound of Formula I was selected as this is ½ the daily MTD of 200 mg/kg. Historical data demonstrated daily dosing of 200 mg/kg yields a TGI of 74% in the ESX030 model on day 28.

Docetaxel was purchased from Selleck in China (Cat. No. S1148) and formulated in 5% DMSO, 30% PEG300, 5% Tween 80 and ddH2O. Docetaxel was dosed IV using 2.5 mpk for Groups 3 and 5, and 5.0 mpk for Groups 4 and 6.

Vehicle preparation for Group 1 (vehicle only) matched that of the compound of Formula (I) formulation. Vehicle was formulated fresh daily and is stable for 24 hours when stored at 4° C.

Results:

Treatment with the vehicle (Group 1) was well tolerated with maximal median BWL of 1% on day 2 of treatment. Tumor volume reach a median of 1986 mm³ on day 36, study termination.

Treatment with 100 mg/kg Formula I compound alone (Group 2) was well tolerated with maximal median BWL of 2% on day 9 of treatment. Tumor volume reach a median of 1710 mm³ on day 57, study termination.

Treatment with 2.5 mg/kg Docetaxel alone (Group 3) was well tolerated with maximal median BWL of 2% on day 9 of treatment. Tumor volume reach a median of 2201 mm³ on day 50, study termination.

Treatment with 5.0 mg/kg Docetaxel alone (Group 4) was well tolerated with maximal median BWL of 1% on day 2. Tumor volume reach a median of 1643 mm³ on day 50, study termination.

Treatment with the combination of the compound of Formula (I) and 2.5 mpk Docetaxel (Group 5) well tolerated with a median BWL of 2% on day 17. Tumor volume reach a median of 1541 mm³ on day 71, study termination.

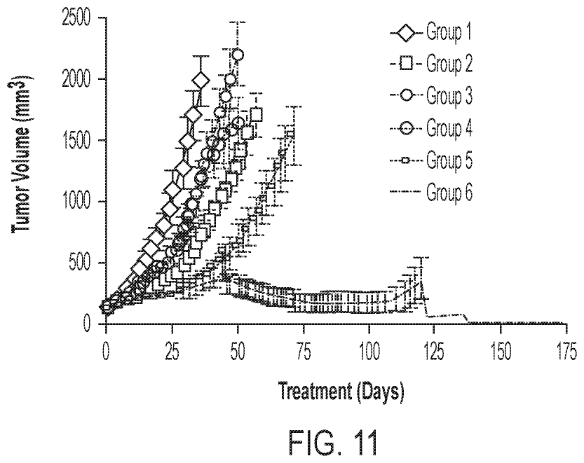

Treatment with the combination of the compound of Formula (I) and 5.0 mpk Docetaxel (Group 6) was overall well tolerated with a median BWL of 2% on day 21. One of 12 animals in this group lost 27% body weight and therefore this animal received the compound of Formula (I) dosing holidays from day 55 to day 60; body weight recovered to 4%. Tumor volume reach a median of 371 mm³ on day 120 (FIG. 11, Table 21). On day 120, three of the twelve animals presented with tumors >1000 mm³ and these 3 animals were removed from the study. The last weekly dose of Docetaxel was delivered on day 134 and the last dose of the compound of Formula (I) was delivered on day 135. On day 136, 3 of the remaining 9 mice displayed tumor volumes of 422 mm³, 146 mm³, 126 mm³, and these mice were removed from the study. The remaining 6 mice were on study were tumor free and remained tumor free until the end of the study on day 155.

The tumor volume results are shown in Table 21 and illustrated in FIG. 11. The combination methodology is described herein and the results are shown in Tables 22 and 23.

TABLE 21
Measured Tumor Volumes and SEM

| Day | | | | | | | | | | | | |
|---|---|---|---|---|---|---|---|---|---|---|---|---|
| (post | Group 1 | | Group 2 | | Group 3 | | Group 4 | | Group 5 | | Group 6 | |
| treatment | Median | | Median | | Median | | Median | | Median | | Median | |
| start) | (SEM) | | (SEM) | | (SEM) | | (SEM) | | (SEM) | | (SEM) | |
| 1 | 140 | (11) | 142 | (12) | 141 | (11) | 142 | (13) | 143 | (11) | 142 | (12) |
| 4 | 199 | (17) | 189 | (19) | 197 | (16) | 185 | (17) | 179 | (13) | 186 | (15) |
| 8 | 284 | (29) | 208 | (26) | 262 | (24) | 220 | (18) | 210 | (20) | 194 | (17) |
| 11 | 362 | (38) | 230 | (32) | 291 | (30) | 236 | (23) | 225 | (22) | 194 | (21) |
| 13 | 453 | (54) | 276 | (40) | 348 | (37) | 279 | (30) | 239 | (26) | 210 | (22) |
| 15 | 521 | (63) | 306 | (50) | 383 | (37) | 325 | (41) | 239 | (27) | 217 | (23) |
| 17 | 602 | (82) | 326 | (50) | 405 | (36) | 382 | (56) | 250 | (27) | 229 | (26) |
| 19 | 690 | (87) | 342 | (51) | 431 | (39) | 431 | (62) | 239 | (32) | 237 | (29) |
| 22 | 802 | (99) | 384 | (55) | 503 | (49) | 472 | (65) | 240 | (33) | 236 | (30) |
| 24 | 941 | (122) | 416 | (54) | 575 | (55) | 521 | (73) | 248 | (38) | 229 | (31) |
| 26 | 1106 | (139) | 436 | (54) | 661 | (70) | 579 | (77) | 260 | (44) | 228 | (33) |
| 29 | 1277 | (154) | 502 | (57) | 775 | (96) | 705 | (101) | 272 | (53) | 228 | (36) |
| 31 | 1502 | (192) | 570 | (63) | 884 | (101) | 835 | (114) | 289 | (59) | 260 | (43) |
| 33 | 1710 | (201) | 631 | (66) | 1002 | (118) | 978 | (139) | 315 | (65) | 300 | (57) |
| 36 | 1986 | (199) | 730 | (83) | 1213 | (150) | 1182 | (158) | 347 | (69) | 329 | (63) |
| 38 | | | 813 | (96) | 1396 | (167) | 1310 | (178) | 362 | (70) | 348 | (67) |
| 40 | | | 870 | (98) | 1493 | (172) | 1373 | (179) | 420 | (84) | 370 | (74) |
| 43 | | | 994 | (112) | 1723 | (200) | 1465 | (195) | 484 | (95) | 382 | (78) |
| 45 | | | 1049 | (116) | 1819 | (207) | 1531 | (208) | 543 | (113) | 380 | (81) |
| 47 | | | 1125 | (126) | 2006 | (242) | 1592 | (212) | 588 | (122) | 347 | (79) |
| 50 | | | 1324 | (142) | 2201 | (260) | 1643 | (211) | 682 | (140) | 325 | (77) |
| 52 | | | 1432 | (148) | | | | | 724 | (140) | 314 | (76) |
| 54 | | | 1563 | (170) | | | | | 792 | (146) | 295 | (77) |
| 57 | | | 1710 | (178) | | | | | 876 | (159) | 281 | (77) |
| 59 | | | | | | | | | 987 | (169) | 263 | (74) |
| 61 | | | | | | | | | 1069 | (182) | 256 | (72) |
| 64 | | | | | | | | | 1157 | (192) | 223 | (64) |
| 66 | | | | | | | | | 1300 | (213) | 222 | (67) |
| 68 | | | | | | | | | 1396 | (224) | 219 | (65) |
| 71 | | | | | | | | | 1541 | (239) | 221 | (68) |
| 73 | | | | | | | | | | | 179 | (60) |
| 75 | | | | | | | | | | | 179 | (60) |
| 78 | | | | | | | | | | | 171 | (55) |
| 80 | | | | | | | | | | | 173 | (54) |
| 82 | | | | | | | | | | | 172 | (54) |
| 85 | | | | | | | | | | | 183 | (59) |
| 87 | | | | | | | | | | | 183 | (60) |
| 89 | | | | | | | | | | | 188 | (68) |
| 92 | | | | | | | | | | | 184 | (68) |
| 94 | | | | | | | | | | | 180 | (72) |
| 96 | | | | | | | | | | | 181 | (74) |
| 99 | | | | | | | | | | | 179 | (75) |
| 101 | | | | | | | | | | | 180 | (77) |
| 103 | | | | | | | | | | | 181 | (77) |
| 106 | | | | | | | | | | | 180 | (79) |
| 110 | | | | | | | | | | | 200 | (91) |
| 113 | | | | | | | | | | | 231 | (108) |
| 115 | | | | | | | | | | | 265 | (122) |
| 117 | | | | | | | | | | | 318 | (147) |
| 120 | | | | | | | | | | | 371 | (171) |
| 122 | | | | | | | | | | | 371 | (171) |
| 124 | | | | | | | | | | | 376 | (171) |
| 127 | | | | | | | | | | | 378 | (170) |
| 129 | | | | | | | | | | | 378 | (171) |
| 131 | | | | | | | | | | | 382 | (170) |
| 134 | | | | | | | | | | | 382 | (170) |

| | |
|---|---|
| 136 | 0 |
| 138 | 0 |
| 141 | 0 |
| 143 | 0 |
| 145 | 0 |
| 148 | 0 |
| 150 | 0 |
| 152 | 0 |
| 155 | 0 |

TABLE 22
Low Dose Docetaxel (2.5 mg/kg) Combination
Statistical Analysis Using Days 1-71

Mean AUC Group 1 = 61.43227
Mean AUC Group 2 = 45.53486
Mean AUC Group 3 = 54.78606
Mean AUC Group 5 = 29.15145
Synergy score: −15.8503185024498
p-value: 0.185489036160427
Combination is additive

TABLE 23
High dose Docetaxel (5.0 mg/kg) Combination
Statistical Analysis Using Days 1-117

Mean AUC Group 1 = 113.65468
Mean AUC Group 2 = 94.51125
Mean AUC Group 4 = 98.12195
Mean AUC Group 6 = −19.23391
Synergy score : −86.41301119467
p-value : 5.50594276980121e−05
Combination is synergistic

### Example 8: Combination of the Compound of Formula (I) and Docetaxel Therapy in a NSCLC PDX Model (LUX001)

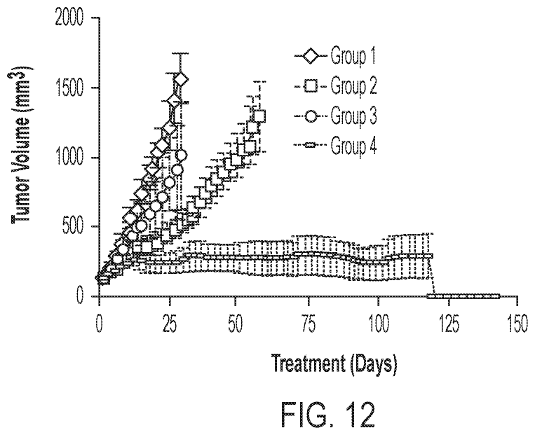

Docetaxel (dosed at 2.5 mg/kg IV on a Q7D schedule) was combined with the compound of Formula I (dosed at 100 mpk PO on a QD schedule) in an MTAP-deficient NSCLC PDX model (LUX001) to evaluate the anti-tumor combination benefit. Each group contained 8 female Nu/Nu mice bearing established LUX001 tumors. Group 1 is the vehicle treated group. Due to BWL with several animals in the compound of Formula (I) groups, dosing holidays were given on days 16-21. In 100 mg/kg of the compound of Formula (I) (Group 2), one animal lost 20% BWL on day 14 and body weight loss (BWL) recovered during the dosing holidays. Group 3 is the docetaxel group. In the compound of Formula (I)+docetaxel combination group (Group 4), one animal reached 20% BWL and was given compound of Formula (I) dosing holidays on days 54-59, 65-73, 77-83, and one animal reached >20% BWL and was given compound of Formula (I) dosing holidays on days 38-46 and a docetaxel dosing holiday on day 42; body weight loss recovered in both these animals. Max mean BWL in this group was 6%. Tumor growth inhibition (methodology described herein) was calculated on day 25 and the compound of Formula (I) (Group 2) yielded a TGI=70%, docetaxel (Group 3) TGI=38% and the combination (Group 4) TGI=91%. Tumor growth curve results are shown in FIG. 12. The combination benefit (methodology described herein) was evaluated on day 120. In the combination group, 4 tumored animals were removed on day 120. The remaining four animals in this group were tumor free when the last dose was delivered on day 114, and these animals remained tumor-free until the arm was terminated on day 141.

### Example 9: Combination of the Compound Shown in Formula (I) and Docetaxel Therapy in a NSCLC PDX Model (LUX034)

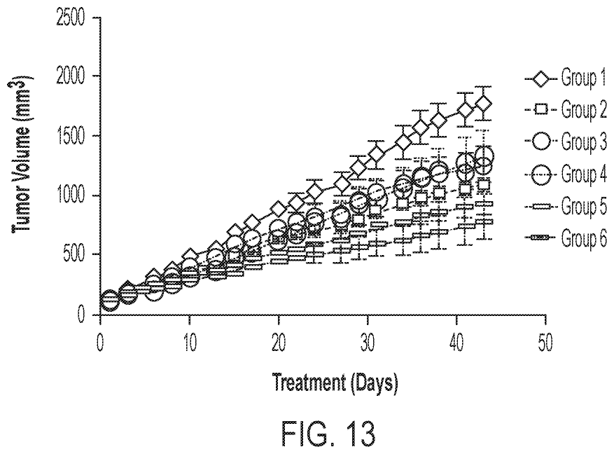

Docetaxel (dosed IV on a Q7D schedule) was combined with the compound of Formula I (dosed at 100 mpk PO on a QD schedule) in an MTAP-deficient NSCLC PDX model (LUX034) to evaluate the anti-tumor combination benefit. Each group contained 8 female Nu/Nu mice bearing established LUX034 tumors. Group 1 is vehicle treated, group 2 is the compound of Formula (I), group 3 is the docetaxel (2.5 mg/kg), group 4 is the docetaxel (5.0 mg/kg), group 5 is the combination of the compound of Formula (I) and docetaxel (2.5 mg/kg), and group 6 is the combination of the compound of Formula (I) and docetaxel (5 mg/kg). All treatments were well tolerated. Tumor growth inhibition (methodology described herein) was calculated on day 43 and the compound of Formula (I) (Group 2) yielded a TGI=41%, docetaxel 2.5 mg/kg (Group 3) TGI=32%, docetaxel 5.0 mg/kg (Group 4) yielded a TGI=27%, combination of the compound of Formula (I)+docetaxel 2.5 mg/kg (Group 5) yielded a TGI=51%, and the combination of the compound of Formula (I)+docetaxel 5.0 mg/kg (Group 6) yielded a TGI=60%. Tumor growth curve results are shown in FIG. 13. The combination benefit (methodology described herein) was evaluated on day 43.

### Example 10: Combination of the Compound of Formula (I) and Docetaxel Therapy in a NSCLC PDX Model (LU6412)

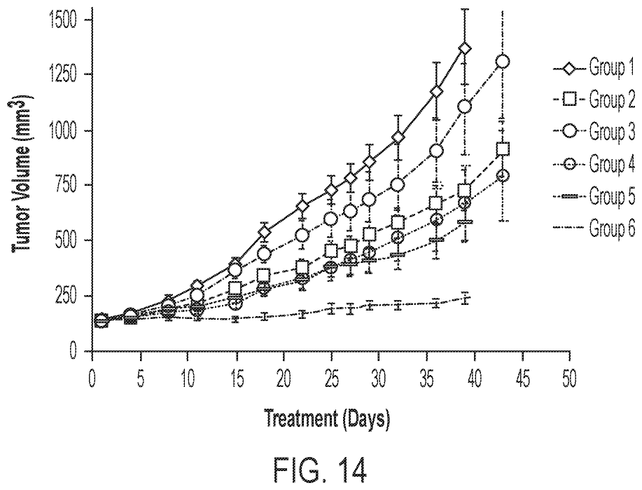

Docetaxel (dosed IV on a Q7D schedule) was combined with the compound of Formula I (dosed at 100 mpk PO on a QD schedule) in an MTAP-deficient NSCLC PDX model (LU6412) to evaluate the anti-tumor combination benefit. Each group contained 8 female BALB/c nude mice bearing established LU6412 tumors. Group 1 is vehicle treated, group 2 is the compound of Formula (I), group 3 is the docetaxel (2.5 mg/kg), group 4 is the docetaxel (5.0 mg/kg), group 5 is the combination of the compound of Formula (I) and docetaxel (2.5 mg/kg), and group 6 is the combination of the compound of Formula (I) and docetaxel (5 mg/kg). All treatments were well tolerated with the following exceptions: 1) groups 4 and 6 received a docetaxel (5 mg/kg) dosing holiday driven by two animals in group 6 approaching 20% BWL, following the dosing holiday BWL rebounded, 2) in group 5 one animal was found dead on day 18, 3) on day 38, one animal in group 8 lost 22.5% body weight and therefore received a dosing holiday for both Formula I compound and docetaxel, and BWL recovered in this animal, and finally 4) in group 6, one animals lost 24% body weight and one animal lost 22% BW, both animals were given dosing holidays for both docetaxel and compound shown in Formula I, BWL did not recover and these two groups were taken down on day 39. Tumor growth inhibition (methodology described herein) was calculated on day 39 and the compound shown in Formula (I) (Group 2) yielded a TGI=52%, docetaxel 2.5 mg/kg (Group 3) TGI=22%, docetaxel 5.0 mg/kg (Group 4) yielded a TGI=57%, combination of the compound shown in Formula (I)+docetaxel 2.5 mg/kg (Group 5) yielded a TGI=64%, and the combination of the compound shown in Formula (I)+docetaxel 5.0 mg/kg (Group 6) yielded a TGI=92%. Tumor growth curve results are shown in FIG. 14. The combination benefit (methodology described herein) was evaluated on day 39.

### Example 11: Combination of the Compound of Formula (I) and Docetaxel Therapy in a NSCLC PDX Model (CTG-1194)

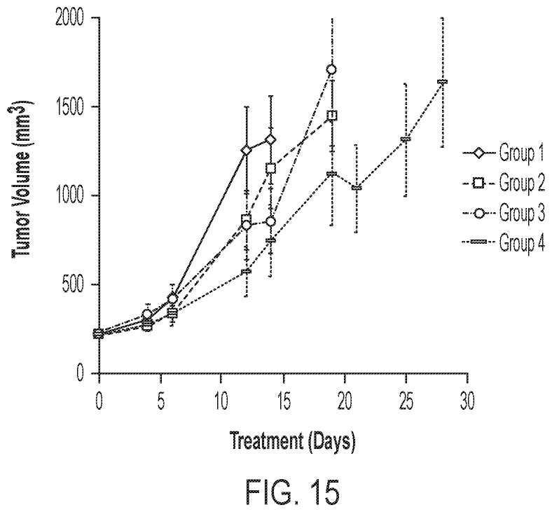

Docetaxel (dosed IV on a Q7D schedule) was combined with the compound shown in Formula I (dosed at 100 mpk PO on a QD schedule) in an MTAP-deficient NSCLC PDX model (CTG-1194) to evaluate the anti-tumor combination benefit. Each group contained 12 female athymic nude mice bearing established CTG-1194 tumors. Group 1 is vehicle treated, group 2 is the compound of Formula (I), group 3 is the docetaxel (5.0 mg/kg), and group 4 is the combination of the compound of Formula (I) and docetaxel (5 mg/kg). All treatments were well tolerated except one animal in group 4 displayed 15% BWL on day 14 and therefore a docetaxel dosing holiday was given on day 14, and BWL did recover. Tumor growth inhibition (methodology described herein) was calculated on day 14 and the compound shown in Formula (I) (Group 2) yielded a TGI=38%, docetaxel 5.0 mg/kg (Group 3) TGI=41%, and the combination of the compound of Formula (I)+docetaxel 5.0 mg/kg (Group 4) yielded a TGI=66%. Tumor growth curve results are shown in FIG. 15. The combination benefit (methodology described herein) was evaluated on day 12.

### Example 12: Combination of the Compound of Formula (I) and Docetaxel Therapy in an Pancreatic PDX Model (PAX001)

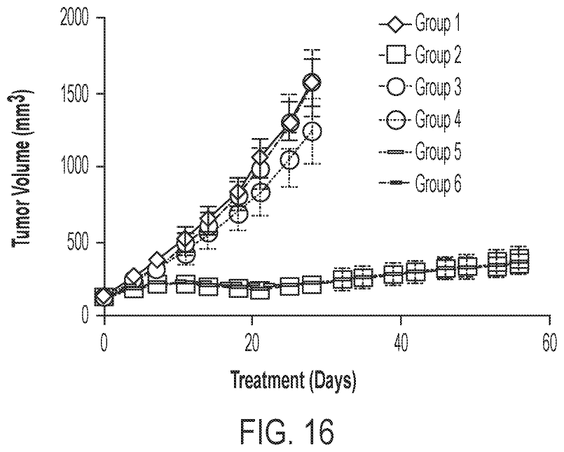

Paclitaxel (dosed IV on a Q7D×2 schedule) was combined with the compound of Formula I (dosed at 100 mpk PO on a QD schedule) in an MTAP-deficient pancreatic PDX model (PAX001) to evaluate the anti-tumor combination benefit. Each group contained 12 female Nu/Nu mice bearing established PAX001 tumors. Group 1 is vehicle treated, group 2 is the compound of Formula (I), group 3 is paclitaxel (5.0 mg/kg), group 4 is paclitaxel (10.0 mg/kg), group 5 is the combination of the compound of Formula (I) and paclitaxel (5 mg/kg), and group 6 is the combination of the compound of Formula (I) and paclitaxel (10 mg/kg). All treatments were well tolerated. Tumor growth inhibition (methodology described herein) was calculated on day 28 and the compound of Formula (I) (Group 2) yielded a TGI=94%, paclitaxel 5 mg/kg (Group 3) TGI=0%, paclitaxel 10.0 mg/kg (Group 4) yielded a TGI=22%, the combination of the compound of Formula (I)+paclitaxel 5.0 mg/kg (Group 5) yielded a TGI=93%, and the combination of the compound of Formula (I)+paclitaxel 10.0 mg/kg (Group 6) yielded a TGI=93%. Tumor growth curve results are shown in FIG. 16. The combination benefit (methodology described herein) was evaluated on day 28.

### Example 13: Combination of the Compound of Formula (I) and Docetaxel Therapy in an Esophageal PDX Model (ES2263)

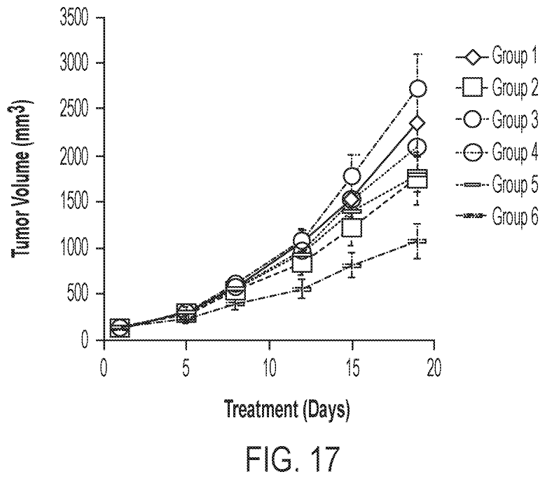

Docetaxel (dosed IV on a Q7D schedule) was combined with the compound of Formula I (dosed at 100 mpk PO on a QD schedule) in an MTAP-deficient esophageal PDX model (ES2263) to evaluate the anti-tumor combination benefit. Each group contained 12 female BALB/c nude mice bearing established ES2263 tumors. Group 1 is the vehicle treated control, group 2 is the compound of Formula (I), group 3 is the docetaxel (2.5 mg/kg), group 4 is the docetaxel (5.0 mg/kg), group 5 is the combination of the compound of Formula (I) and docetaxel (2.5 mg/kg), and group 6 is the combination of the compound of Formula (I) and docetaxel (5 mg/kg). All treatments were well tolerated except one animal in group 5 was found dead on day 8 and one animal in group 6 was found dead on day 19. Tumor growth inhibition (methodology described herein) was calculated on day 19 and the compound of Formula (I) (Group 2) yielded a TGI=27%, docetaxel 2.5 mg/kg (Group 3) TGI=−17%, docetaxel 5.0 mg/kg (Group 4) yielded a TGI=12%, combination of the compound of Formula (I)+docetaxel 2.5 mg/kg (Group 5) yielded a TGI=25%, and the combination of the compound of Formula (I)+docetaxel 5.0 mg/kg (Group 6) yielded a TGI=57%. Tumor growth curve results are shown in FIG. 17. The combination benefit (methodology described herein) was evaluated on day 19.

### Example 14: Combination of the Compound of Formula (I) and Gemcitabine Therapy in Pancreatic PAX041 PDX Model

Study Objective: The objective of this study was to evaluate the efficacy of the compound of Formula (I), given once daily (PO) alone and in combination with Gemcitabine, against an established MTAP-deficient patient derived xenograft tumors (PDX), PAX041, in female mice.

Study Design: The study mice were randomized on Day 23 post inoculation into four study groups based on a median tumor volume of 133 mm³. Treatment began on Day 23 post inoculation (first day of treatment denoted as day 1) with the treatment schedules summarized in Table 24.

TABLE 24
Study Design/Treatment Schedules

| | # of | | | |
|---|---|---|---|---|
| Group | Animals | Treatment | Route | Dose and Schedule |
| 1 | 12 | Vehicle Control | PO | QD × 28 days |
| 2 | 12 | The Compound of | PO | 100 mpk QD × 28 days |
| | | Formula (I) | | |
| 3 | 12 | Gemcitabine | IP | 20 mpk Q3D × 10 days |
| 4 | 12 | The compound of | PO + | 100 mpk QD × 28 days |
| | | Formula (I) + | IP | 20 pk Q3D × 10 days |
| | | Gemcitabine | | |
| | | (simultaneous | | |
| | | treatment) | | |

Materials and Methods: Female Nu/Nu mice weighing 18-22 g were purchased from Beijing Vital River Laboratory Animal Technology Co. Ltd. (Beijing, China) and subcutaneously implanted with PAX041 tumor fragments. PAX041 is an MTAP-deficient human primary pancreatic cancer xenograft model established at ChemPartner.

Tumor volumes were measured twice weekly in two dimensions using a caliper, and the volume was expressed in mm³ using the formula: V=(L×W×W)/2, where V is tumor volume, L is tumor length (the longest tumor dimension) and W is tumor width (perpendicular to L). Tumor growth inhibition rate (TGI %) of each dosing group was calculated according to the following formula: TGI %=[1−(TVi−TV0)/(TVvi−TVv0)]×100%; TVi is average tumor volume of a dosing group on a specific day; TV0 is average tumor volume of a dosing group on the initial day; TVvi is average tumor volume of the vehicle group on a specific day; TVv0 is average tumor volume of the vehicle group on the initial day. Body weights were measured twice per week.

The compound of Formula (I) supplied as a formulation of amorphous Formula (I). The compound was stored at 4° C. protected from light. the compound of Formula (I) was formulated daily in a vehicle. Formulated, the compound of Formula (I) is stable for 24 hours when stored at 4° C. protected from light.

The compound of Formula (I) was dosed orally at 100 mg/kg, daily, for Groups 2 and 4.

Gemcitabine was purchased from Selleck in China (Cat. No. S1149) and formulated in sterile saline. Gemcitabine was dosed IP using 20 mpk for Groups 3 and 4. Vehicle preparation for Group 1 (vehicle only) matched that of the compound of Formula (I) formulation. Vehicle was formulated fresh daily and is stable for 24 hours when stored at 4° C.

### Results:

Treatment with the vehicle (Group 1) was well tolerated with 0 body weight loss (BWL) during the study. Tumor volume reach a median of 847 mm³ on day 28, study termination.

Treatment with 100 mp/kg of the compound of Formula (I) alone (Group 2) was well tolerated with maximal median BWL of 3% on day 12 of treatment. Tumor volume reach a median of 461 mm³ on day 28 (TGI=55%), study termination.

Treatment with 20 mp/kg Gemcitabine alone (Group 3) was well tolerated with no median BWL throughout the study. Tumor volume reach a median of 728 mm³ on day 28 (TGI=16%), study termination.

Treatment with the combination of the compound of Formula (I) and Gemcitabine (Group 4) was well tolerated with a median BWL of 3% on day 24. Tumor volume reach a median of 357 mm³ on day 28 (TGI=69%), study termination.

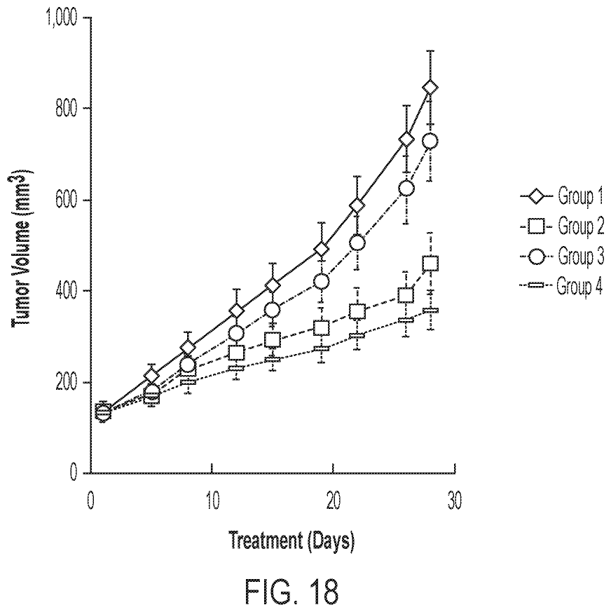

The tumor volume results of Table 25 are illustrated in FIG. 18. The combination benefit (methodology described herein) was evaluated using data on day 1-26, results of this analysis are shown in Table 26.

TABLE 25
Measured Tumor Volumes and SEM

| Day (post | Group 1 | | Group 2 | | Group 3 | | Group 4 | |
|---|---|---|---|---|---|---|---|---|
| treatment start) | Median (SEM) | | Median (SEM) | | Median (SEM) | | Median (SEM) | |
| 1 | 133.59 | (17.55) | 136.46 | (20.36) | 130.73 | (18.10) | 132.53 | (17.47) |
| 5 | 213.56 | (25.22) | 171.99 | (26.20) | 180.92 | (23.27) | 167.01 | (20.15) |
| 8 | 274.53 | (36.44) | 229.40 | (29.53) | 238.21 | (25.14) | 199.62 | (23.08) |
| 12 | 355.95 | (48.03) | 264.21 | (32.19) | 308.15 | (36.89) | 229.58 | (24.62) |
| 15 | 412.91 | (47.07) | 294 | (35.31) | 359.28 | (38.26) | 249.01 | (24.88) |
| 19 | 493.47 | (58.19) | 319.51 | (42.90) | 420.45 | (46.58) | 274.80 | (30.58) |
| 22 | 588.02 | (64.77) | 356.12 | (50.74) | 505.47 | (59.31) | 303.70 | (33.27) |
| 26 | 733.22 | (73.05) | 389.77 | (53.38) | 623.25 | (74.71) | 336.98 | (38.91) |
| 28 | 847.34 | (79.74) | 460.86 | (68.22) | 728.00 | (87.33) | 357.25 | (42.38) |

TABLE 26
Combination Statistical Analysis for days 0-26

Mean AUC Group 1 = 10.694597
Mean AUC Group 2 = 7.152290
Mean AUC Group 3 = 9.565932
Mean AUC Group 4 = 6.235600
In vivo Synergy Score: 1.98207582213063
p-value: 0.892547602901707

### Example 15: Combination of the Compound of Formula (I) and Gemcitabine Therapy in a Pancreatic PDX Model (PAX001)

Study Objective: The objective of this study was to evaluate the efficacy of the compound of Formula (I), given once daily (PO) alone and in combination with Gemcitabine, against an established MTAP-deficient patient derived xenograft tumors (PDX), PAX001, in female mice.

Study Design: The study mice were randomized on Day 18 post inoculation into four study groups based on a median tumor volume of 188 mm³. Treatment began on Day 18 post inoculation (first day of treatment denoted as day 1) with the treatment schedules summarized in Table 27.

TABLE 27
Study Design/Treatment Schedules

| | # of | | | |
|---|---|---|---|---|
| Group | Animals | Treatment | Route | Dose and Schedule |
| 1 | 12 | Vehicle Control | PO | QD × 21 days |
| 2 | 12 | The Compound of | PO | 100 mpk QD × 21 days |
| | | Formula (I) | | |
| 3 | 12 | Gemcitabine | IP | 20 mpk Q3D × 7 days |
| 4 | 12 | The compound of | PO + | 100 mpk QD × 21 days |
| | | Formula (I) + | IP | 20 pk Q3D × 7 days |
| | | Gemcitabine | | |
| | | (simultaneous | | |
| | | treatment) | | |

Materials and Methods: Female Nu/Nu mice weighing 18-22 g were purchased from Beijing Vital River Laboratory Animal Technology Co. Ltd. (Beijing, China) and subcutaneously implanted with PAX001 tumor fragments. PAX001 is an MTAP-deficient human primary pancreatic cancer xenograft model established at ChemPartner.

Tumor volumes were measured twice weekly in two dimensions using a caliper, and the volume was expressed in mm³ using the formula: V=(L×W×W)/2, where V is tumor volume, L is tumor length (the longest tumor dimension) and W is tumor width (perpendicular to L). Tumor growth inhibition rate (TGI %) of each dosing group was calculated according to the following formula: TGI %=[1−(TVi−TV0)/(TVvi−TVv0)]×100%; TVi is average tumor volume of a dosing group on a specific day; TV0 is average tumor volume of a dosing group on the initial day; TVvi is average tumor volume of the vehicle group on a specific day; TVv0 is average tumor volume of the vehicle group on the initial day. Body weights were measured twice per week.

The compound of Formula (I) was supplied as a formulation comprising amorphous Formula (I). The compound was stored at 4° C. protected from light. the compound of Formula (I) was formulated daily in a vehicle. Formulated, the compound of Formula (I) is stable for 24 hours when stored at 4° C. protected from light.

The compound of Formula (I) was dosed orally at 100 mg/kg, daily, for Groups 2 and 4.

Gemcitabine was purchased from Selleck in China (Cat. No. S1149) and formulated in sterile saline. Gemcitabine was dosed IP using 20 mpk for Groups 3 and 4.

Vehicle preparation for Group 1 (vehicle only) matched that of the compound of Formula (I) formulation. Vehicle was formulated fresh daily and is stable for 24 hours when stored at 4° C.

### Results:

Treatment with the vehicle (Group 1) was well tolerated with maximal median BWL of 2% on day 2 of treatment. Tumor volume reach a median of 965 mm³ on day 21, study termination.

Treatment with 100 mp/kg the compound of Formula (I) alone (Group 2) was well tolerated with maximal median BWL of 7% on day 14 of treatment. Tumor volume reach a median of 320 mm³ on day 21 (TGI=83%), study termination.

Treatment with 10 mp/kg Gemcitabine alone was well tolerated with a maximal median BWL of 5% on day 8. Tumor volume reach a median of 529 mm³ on day 21 (TGI=56%), study termination.

Treatment with the combination of the compound of Formula (I) and Gemcitabine was well tolerated with a median BWL of 5% on day 9. Tumor volume reach a median of 274 mm³ on day 21 (TGI=89%), study termination.

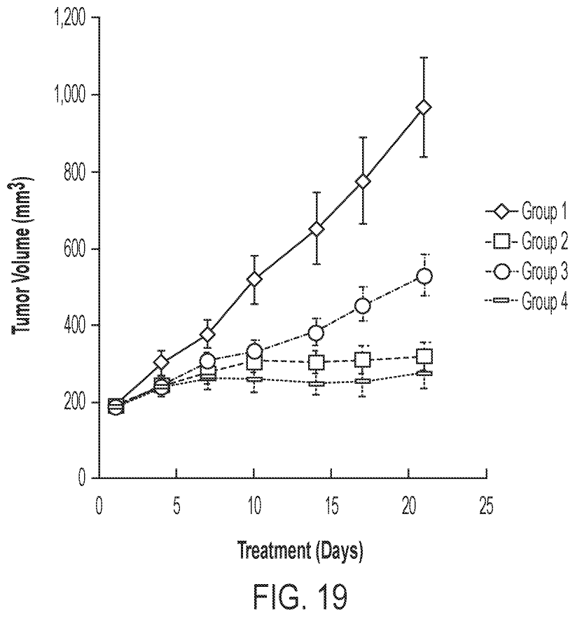

Tumor volumes from each group are shown in Table 28 are illustrated in FIG. 19. The combination benefit (methodology described herein) was evaluated using data from days 0-21. Combination results are shown in Table 29.

TABLE 28
Measured Tumor Volumes and SEM

| Day (post | Group 1 | | Group 2 | | Group 3 | | Group 4 | |
|---|---|---|---|---|---|---|---|---|
| treatment start) | Median (SEM) | | Median (SEM) | | Median (SEM) | | Median (SEM) | |
| 1 | 189.63 | (12.67) | 189.33 | (12.19) | 187.17 | (12.78) | 187.79 | (12.24) |
| 4 | 302.53 | (31.92) | 243.82 | (16.22) | 239.38 | (17.76) | 239.54 | (25.83) |
| 7 | 377.06 | (37.59) | 275.39 | (28.63) | 308.93 | (19.84) | 264.09 | (30.27) |
| 10 | 519.97 | (63.74) | 305.37 | (28.65) | 331.79 | (29.21) | 258.68 | (34.01) |
| 14 | 651.06 | (93.91) | 303.34 | (30.54) | 381.40 | (35.41) | 250.15 | (33.52) |
| 17 | 776.03 | (110.94) | 310.62 | (35.80) | 454.64 | (45.32) | 255.44 | (34.93) |
| 21 | 965.37 | (129.29) | 319.73 | (37.98) | 529.15 | (56.00) | 274.21 | (41.47) |

TABLE 29
Combination Statistical Analysis for days 0-21

Mean AUC Group 1 = 7.949802
Mean AUC Group 2 = 3.194364
Mean AUC Group 3 = 5.059819
Mean AUC Group 4 = 2.062083
In vivo Synergy Score: 22.110013418178
p-value: 0.0976586381730211

### Example 16: Combination of the Compound of Formula (I) and Gemcitabine Therapy in a Pancreatic KP4 Model

Study Objective: The objective of this study was to evaluate the potential efficacy of the compound of Formula (I) given once daily (PO), alone and in combination with Gemcitabine, against established MTAP-deficient pancreatic xenograft tumors (KP4), in female mice.

Study Design: 5-6 weeks old female, CB-17 SCID mice were subcutaneously inoculated with 1×10⁷ KP4 cells in serum free media+Matrigel (1:1). The mice were randomized on Day 26 once tumors averaged 200 mm3, into treatment groups and dosed as outlined in Table 30 below.

TABLE 30
Study Design/Treatment Schedules

| | # of | | | |
|---|---|---|---|---|
| Group | Animals | Treatment | Route | Dose and Schedule |
| 1 | 12 | Vehicle#1: 0.9% | IV + | 5 mL/kg Q7D × 4 |
| | | NaCl + | PO | 10 mL/kg QD × 29 |
| | | Vehicle#2: | | |
| 2 | 12 | The compound of | PO | 100 mg/kg QD × 29 |
| | | Formula (I) | | |
| 3 | 12 | Gemcitabine | IP | 20 mg/kg Q3D × 10 |
| 4 | 12 | The compound of | PO + | 100 mg/kg QD × 29 |
| | | Formula (I) + | IP | 20 g/kg Q3D × 10 |
| | | Gemcitabine | | |
| | | (simultaneous | | |
| | | treatment) | | |

Materials and Methods: Tumor volumes were measured twice weekly in two dimensions using a caliper, and the volume was expressed in mm³ using the formula: V=(L×W×W)/2, where V is tumor volume, L is tumor length (the longest tumor dimension) and W is tumor width (perpendicular to L). Body weights were measured twice per week.

The compound of Formula (I) was supplied as a formulation comprising amorphous Formula (I). The compound was stored at 4° C. protected from light. The compound of Formula (I) was formulated daily in a vehicle. Formulated, the compound of Formula (I) is stable for 24 hours when stored at 4° C. protected from light.

The compound of Formula (I) was dosed orally at 100 mg/kg, daily, for Groups 2 and 4.

Gemcitabine was purchased from Myoderm (Cat. No. 00002-7501-01) and formulated in 0.9% NaCl for sterile injection. Gemcitabine was dosed IP using 20 mg/kg for groups 3 and 4.

Vehicle preparation for Group 1 (vehicle only) matched that of the compound of Formula (I) formulation. Vehicle was formulated fresh daily and is stable for 24 hours when stored at 4° C.

### Results:

Treatment with the vehicle was well tolerated with 0 body weight loss (BWL) during the study. Tumor volume reach a median of 1687.4 mm³ on day 19 of treatment, Group 1 termination.

Treatment with 100 mp/kg the compound of Formula (I) alone (Group 2) was well tolerated with maximal median BWL of 3% on day 4 of treatment. Tumor volume reach a median of 1426.7 mm³ on day 36 of treatment, Group 2 termination.

Treatment with 20 mp/kg Gemcitabine alone was well tolerated with a maximal median BWL of 3% on day 3 of treatment. Tumor volume reach a median of 1318.61 mm³ on day 22 of treatment, Group 3 termination.

Treatment with the combination of 100 mg/kg the compound of Formula (I) and 20 mg/kg Gemcitabine was well tolerated with a median BWL of 5% on day 10 of treatment. Tumor volume reach a median of 1284.3 mm³ on day 36 of treatment, Group 4 termination.

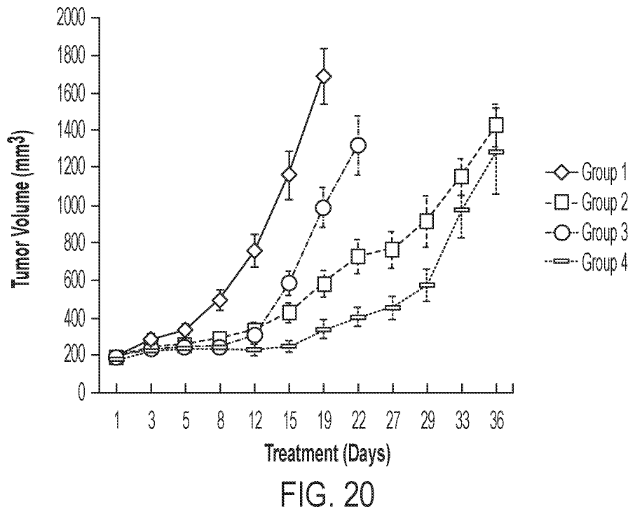

Tumor volume results from each group are shown in Table 31 are illustrated in FIG. 20. The combination benefit (methodology described herein) was evaluated using data from days 0-19. Combination results are shown in Table 32.

TABLE 31
Measured Tumor Volumes and SEM

| Day (post | Group 1 | | Group 2 | | Group 3 | | Group 4 | |
|---|---|---|---|---|---|---|---|---|
| treatment start) | Median (SEM) | | Median (SEM) | | Median (SEM) | | Median (SEM) | |
| 1 | 184.6 | (15.9) | 186.1 | (17.0) | 187.5 | (16.5) | 178.6 | (21.4) |
| 3 | 281.6 | (30.1) | 245.6 | (18.7) | 239.2 | (17.0) | 221.8 | (23.5) |
| 5 | 335.2 | (29.2) | 251.2 | (22.3) | 238.7 | (16.7) | 233.7 | (28.9) |
| 8 | 494.3 | (55.9) | 288.1 | (27.6) | 246.3 | (18.7) | 239.1 | (27.3) |
| 12 | 755.7 | (84.0) | 330.8 | (42.5) | 307.8 | (23.3) | 229.7 | (32.0) |
| 15 | 1159.8 | (128.9) | 426.1 | (52.4) | 584.3 | (60.7) | 245.6 | (30.8) |
| 19 | 1687.4 | (147.3) | 582.8 | (68.9) | 986.8 | (108.7) | 337.4 | (49.6) |
| 22 | | | 724.9 | (91.3) | 1318.6 | (156.0) | 400.6 | (51.6) |
| 26 | | | 557.7 | (59.1) | | | 398.7 | (61.3) |
| 27 | | | 759.8 | (99.9) | | | 451.1 | (61.8) |
| 29 | | | 911.1 | (133.6) | | | 572.5 | (85.2) |
| 33 | | | 1150.9 | (100.9) | | | 974.9 | (150.9) |
| 36 | | | 1426.7 | (113.5) | | | 1284.3 | (229.8) |

TABLE 32
Combination Statistical Analysis for days 0-19

Mean AUC Group 1 = 8.591087
Mean AUC Group 2 = 3.361968
Mean AUC Group 3 = 4.831409
Mean AUC Group 4 = 1.048925
In vivo Synergy Score: 16.8387840902352
p value: 0.154667715778082

### Example 17: Combination of the Compound of Formula (I) and Gemcitabine Therapy in a NSCLC PDX Model

Gemcitabine (IP 20 mpk on days 1, 4, 7, 10, and 13) was combined with the compound of Formula (I) (PO 100 mpk for 38 days) in an MTAP-deficient NSCLC PDX model (LU1513) to evaluate the anti-tumor combination benefit. On day 11 of the experiment one of 12 animals in the combination group lost 28% of its pre-therapy body weight. This combination was not well-tolerated (in this model), precluding anti-tumor combination benefit evaluation.

### Example 18: Combination of the Compound of Formula (I) and Gemcitabine Therapy in a NSCLC PDX Model (LU6431)

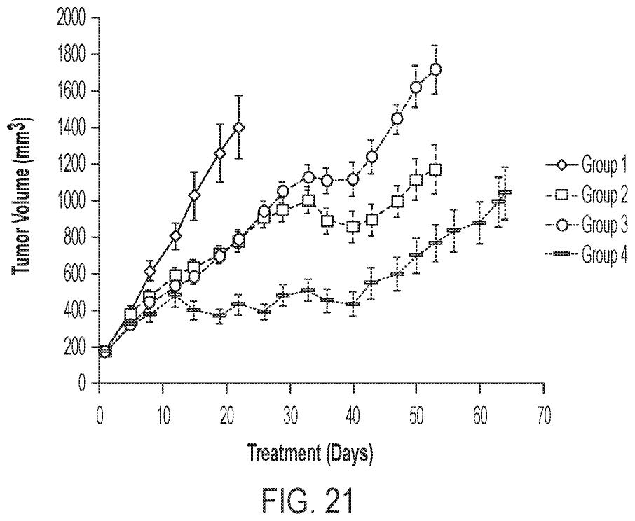

Gemcitabine (dosed IP on a Q3D schedule) was combined with the compound of Formula I (dosed at 100 mpk PO on a QD schedule) in an MTAP-deficient NSCLC PDX model (LU6431) to evaluate the anti-tumor combination benefit. Each group contained 12 female BALB/c mice bearing established LU6431 tumors. Group 1 is vehicle treated, group 2 is the compound shown in Formula (I), group 3 is gemcitabine (20.0 mg/kg), and group 4 is the combination of gemcitabine (20.0 mg/kg) and the compound of Formula (I). All treatments were well tolerated. Tumor growth inhibition (methodology described herein) was calculated on day 22 and the compound of Formula (I) (Group 2) yielded a TGI=45%, gemcitabine 20 mg/kg (Group 3) yielded a TGI=43%, and the combination of the compound of Formula (I)+gemcitabine 20.0 mg/kg (Group 4) yielded a TGI=69%. Tumor growth curve results are shown in FIG. 21. The combination benefit (methodology described herein) was evaluated on day 22.

### Example 19: Combination of the Compound of Formula (I) and Paclitaxel Therapy in a NSCLC PDX Model

Paclitaxel (IP 15 mpk on days 1, 8, 15 and 38) was combined with the compound of Formula (I) (PO 100 mpk for 38 days) in an MTAP-deficient NSCLC PDX model (LU1513) to evaluate the anti-tumor combination benefit. The LU1513 model was found to be resistant to Paclitaxel with a TGI=−6%. Consistent with this observation, the TGI of single agent the compound of Formula (I) (74%) was similar to the combination TGI (78%). Paclitaxel resistance in this model precluded combination benefit evaluation.

All publications, patents and patent applications cited in this specification are incorporated herein by reference for the teaching to which such citation is used.

The specific responses observed may vary according to and depending on the dosing of the particular active compound or combination selected, as well as the type of formulation and mode of administration employed, and such expected variations or differences in the results are contemplated in accordance with practice of the present invention.

Although specific embodiments of the present invention are herein illustrated and described in detail, the invention is not limited thereto. The above detailed descriptions are provided as exemplary of the present invention and should not be construed as constituting any limitation of the invention. Modifications will be obvious to those skilled in the art, and all modifications that do not depart from the spirit of the invention are intended to be included with the scope of the appended claims.

1. A method for the treatment of MTAP-deficient non-small cell lung cancer (NSCLC) or pancreatic cancer in a patient in need thereof, comprising administering:
(a) a therapeutically effective amount of a compound of Formula (I): 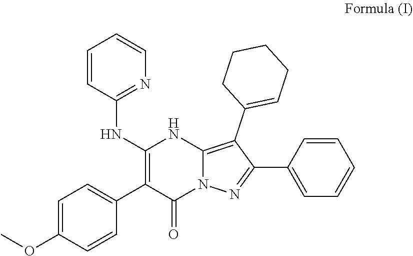 [SMILES: COc1ccc(-c2c(Nc3ccccn3)[nH]c3c(C4=CCCCC4)c(-c4ccccc4)nn3c2=O)cc1]
or a pharmaceutically acceptable salt thereof, and
(b) a therapeutically effective amount of a taxane.

2. (canceled)

3. The method of claim 1, wherein the taxane is docetaxel, paclitaxel, or nab-paclitaxel.

4. The method of claim 3, wherein the taxane is docetaxel.

5. The method of claim 1, further comprising administering one or more additional therapeutic agents.

6. (canceled)

7. The method of claim 5, wherein the additional therapeutic agent is a platinum-based chemotherapeutic.

8. The method of claim 7, wherein the platinum-based chemotherapeutic is cisplatin, carboplatin, oxaliplatin, nedaplatin, triplatin tetra nitrate, phenanthriplatin, picoplatin, or satraplatin.

9. The method of claim 7, wherein the platinum-based chemotherapeutic is carboplatin or cisplatin.

10. The method of claim 1, wherein the patient failed to respond, ceased responding, or experienced disease progression after one or more prior lines of therapy for treating MTAP-deficient NSCLC or pancreatic cancer.

11. The method of claim 10, wherein the compound of Formula (I) or a pharmaceutically acceptable salt thereof is a second line of therapy for treating MTAP-deficient NSCLC or pancreatic cancer.

12. The method of claim 10, wherein the compound of Formula (I) or a pharmaceutically acceptable salt thereof is a third line of therapy for treating MTAP-deficient NSCLC or pancreatic cancer.

13. The method of claim 1, wherein the MTAP-deficient NSCLC or pancreatic cancer is newly diagnosed.

14. The method of claim 1, wherein the amount of the compound of Formula (I) or a pharmaceutically acceptable salt thereof is about 20 mg to about 800 mg.

15. The method of claim 1, wherein the amount of the compound of Formula (I) or a pharmaceutically acceptable salt thereof is once or twice daily dosing.

16. The method of claim 1, wherein the compound of Formula (I) or a pharmaceutically acceptable salt thereof is administered orally.

17-19. (canceled)

20. The method of claim 3, wherein the taxane is nab-paclitaxel.

21. The method of claim 1, further comprising administering a therapeutically effective amount of a DNA synthesis inhibitor.

22. (canceled)

23. The method claim 21, wherein the DNA synthesis inhibitor is gemcitabine.

24-30. (canceled)

31. The method of claim 1, wherein the pancreatic cancer is pancreatic ductal adenocarcinoma (PDAC).

32. The method of claim 1, wherein the pancreatic cancer is unresected, locally advanced or metastatic.

33. The method of claim 1, wherein the compound of Formula (I) or a pharmaceutically acceptable salt thereof and the taxane are administered concurrently.

34. The method of claim 1, wherein the compound of Formula (I) or a pharmaceutically acceptable salt thereof and the taxane are administered sequentially.
